(19) United States
(12) Patent Application Publication
(10) Pub. No.: US 2020/0165354 A1
(43) Pub. Date: May 28, 2020

(54) Novel Anti-PCSK9 Antibodies

(71) Applicant: WuXi Biologics Ireland Limited, Dublin (IE)

(72) Inventors: Jieying LIU, Shanghai (CN); Jing LI, Lexington, MA (US); Gennady GOLOLOBOV, Gaithersburg, MD (US)

(21) Appl. No.: 16/334,774

(22) PCT Filed: Sep. 12, 2017
(86) PCT No.: PCT/CN2017/101356

§ 371 Date: Mar. 20, 2019

(30) Foreign Application Priority Data
Sep. 20, 2016 (CN) PCT/CN2016/099492

(51) Int. Cl. C07K 16/40; A61P 3/06; A61P 9/10

(52) U.S. Cl. CPC C07K 16/40; A61K 2039/505; A61P 9/10; A61P 3/06

(57) ABSTRACT

The present disclosure provides monoclonal antibodies against Proprotein Convertase Subtilisin/Kexin type 9 (PCSK9), which can block the binding of PCSK9 to LDL receptor, and therefore lower the level LDL-C. The antibodies of disclosure provide very potent agents for the treatment of multiple CVDs.

### FIELD OF THE INVENTION

The present disclosure generally relates to novel anti-PCSK9 antibodies.

### BACKGROUND

Cardiovascular diseases (CVD) remains the number one cause of death globally (World Health Organization (WHO), 2011. World Health Organization). Various studies have shown that lowering the low-density lipoprotein cholesterol (LDL-C) reduces the risk of CVD. There is a significantly medical need for CVD despite the treatment with statins, the current first choice of lipid lowering agents. A significant portion of patients is either unable to tolerate satisfactory doses or fail to achieve lipid control on statin therapy (Baigent, C. et al., Lancet 2000, 376(9753), 1670-1681).

Proprotein Convertase Subtilisin/Kexin type 9 (PCSK9) was originally discovered as neural apoptosis regulated convertase-1. It is primarily synthesized in the small intestine and liver (Seidah N G et al., Proc Natl Acad Sci USA 2003; 100:928-33). Mature PCSK9 is secreted from the liver cells after intracellular autocatalytic cleavage of its prodomain (McNutt, M. C. et al., J. Biol. Chem. 2007, 20(282), 20799-20803). The important role of PCSK9 in regulating cholesterol metabolism was firstly found by the recognition of two gain-of function mutations in PCSK9 in two French families with autosomal dominant hypercholesterolemia (Abifadel M et al., Nat Genet 2003; 34:154-6). PCSK9 regulates cholesterol metabolism mainly by binding to the low-density lipoprotein receptor (LDLR) for degradation in liver. In the absence of PCSK9, the hepatic LDLR is recycled back to the cell membrane after delivering LDL-C to the lysozyme for degradation. Binding of PCSK9 and LDLR prevents the normal recycling of LDLR and instead enhances the LDLR degradation (Verbeek, R., et al., Eur J Pharmacol 2015; Lo Surdo P et al., EMBO Rep 2011; 12:1300-5).

Several therapeutic approaches to inhibit PCSK9 are in development, including direct inhibition of PCSK9 binding to LDLR by antibody or peptides; inhibition of PCSK9 synthesis by gene silencing agents and inhibition of PCSK9 intracellular production by small molecules (MICHEL FARNIER, ARCHIVES OF CARDIOVASCULAR DISEASE, 2014, 107, 58-66). The recently approved monoclonal antibodies Alirocumab and Evolocumab have shown promising efficacy of LDL-C reduction in phase II and phase III clinical studies. Current evidence shows up to 70% reduction in LDL-C levels independent of background Statin therapy (Dias, C. S et al., J. Am. Coll. Cardiol. 2012, 60(19), 1888-1898; Giugliano, R. P et al., Lancet, 2012, 380(9858), 2007-2017; McKenney, J. M et al., J. Am. Coll. Cardiol. 2012, 59(25), 2344-2353).

### BRIEF SUMMARY OF THE INVENTION

The present disclosure provides novel monoclonal anti-PCSK9 antibodies (in particular humanized antibodies), polynucleotides encoding the same, and methods of using the same.

In one aspect, the present disclosure provides isolated monoclonal antibodies or antigen binding fragments thereof, comprising a heavy chain CDR sequences selected from the group consisting of: SEQ ID NOs: 1, 3, 5, 13, 15, and 17.

In certain embodiments, the antibodies or antigen binding fragments thereof comprises a light chain CDR sequences selected from the group consisting of: SEQ ID NOs: 7, 9, 11, 19, 21, 23 and 25.

In certain embodiments, the antibodies or antigen binding fragments thereof comprises a heavy chain variable region selected from the group consisting of:

a) a heavy chain variable region comprising SEQ ID NO: 1, SEQ ID NO: 3, and/or SEQ ID NO: 5; and

b) a heavy chain variable region comprising SEQ ID NO: 13, SEQ ID NO: 15, and/or SEQ ID NO: 17.

In certain embodiments, the antibodies or antigen binding fragments thereof comprises a light chain variable region selected from the group consisting of:

a) a light chain variable region comprising SEQ ID NO: 7, SEQ ID NO: 9, and/or SEQ ID NO: 11;

b) a light chain variable region comprising SEQ ID NO: 19, SEQ ID NO: 21, and/or SEQ ID NO: 23; and

c) a light chain variable region comprising SEQ ID NO: 25, SEQ ID NO: 21, and/or SEQ ID NO: 23.

In certain embodiments, the antibodies or antigen binding fragments thereof comprises:

a) a heavy chain variable region comprising SEQ ID NO: 1, SEQ ID NO: 3, and/or SEQ ID NO: 5; and a light chain variable region comprising SEQ ID NO: 7, SEQ ID NO: 9, and/or SEQ ID NO: 11;

b) a heavy chain variable region comprising SEQ ID NO: 13, SEQ ID NO: 15, and/or SEQ ID NO: 17; and a light chain variable region comprising SEQ ID NO: 19, SEQ ID NO: 21, and/or SEQ ID NO: 23; or

c) a heavy chain variable region comprising SEQ ID NO: 13, SEQ ID NO: 15, and/or SEQ ID NO: 17; and a light chain variable region comprising SEQ ID NO: 25, SEQ ID NO: 21, and/or SEQ ID NO: 23.

In certain embodiments, the antibodies or antigen binding fragments thereof comprises a heavy chain variable region selected from the group consisting of: SEQ ID NO: 36, SEQ ID NO: 40, SEQ ID NO: 44, SEQ ID NO: 48 and the homologue sequences of at least 80% (e.g. at least 85%, 88%, 90%, 91%, 92%, 93%, 94%, 95%, 96%, 97%, 98% or 99%) sequence identity thereof.

In certain embodiments, the antibodies or antigen binding fragments thereof comprises a light chain variable region selected from the group consisting of: SEQ ID NO: 38, SEQ ID NO: 42, SEQ ID NO: 46, SEQ ID NO: 50 and the homologue sequences of at least 80% (e.g. at least 85%, 88%, 90%, 91%, 92%, 93%, 94%, 95%, 96%, 97%, 98% or 99%) sequence identity thereof.

In certain embodiments, the antibodies or antigen binding fragments thereof comprises:

a) a heavy chain variable region comprising SEQ ID NO: 36 and a light chain variable region comprising SEQ ID NO: 38;

b) a heavy chain variable region comprising SEQ ID NO: 40 and a light chain variable region comprising SEQ ID NO: 42;

c) a heavy chain variable region comprising SEQ ID NO: 44 and a light chain variable region comprising SEQ ID NO: 46;

d) a heavy chain variable region comprising SEQ ID NO: 48 and a light chain variable region comprising SEQ ID NO: 50; or

e) a heavy chain variable region and a light chain variable region of at least 80% (e.g. at least 85%, 88%, 90%, 91%, 92%, 93%, 94%, 95%, 96%, 97%, 98% or 99%) sequence identity to a), b), c) or d).

In certain embodiments, the antibodies or antigen binding fragments thereof is capable of specifically binding to human PCSK9 at a KD value no more than 10⁻⁷ M, no more than 10⁻⁸ M, no more than 10⁻⁹ M, or no more than 10⁻¹⁰ M, no more than 10⁻¹¹ M, no more than 10⁻¹² M, as measured by surface plasmon resonance (SPR) binding assay.

In certain embodiments, the antibodies or antigen binding fragments thereof is capable of specifically binding to human PCSK9 at a KD value no more than 10⁻⁷ M, no more than 10⁻⁸ M, no more than 10⁻⁹ M, no more than 10⁻¹⁰ M, no more than 10⁻¹¹ M, no more than 10⁻¹² M, as measured by ELISA assay.

In certain embodiments, the antibodies or antigen binding fragments thereof binds to monkey PCSK9 at a KD value no more than 10⁻⁷ M, no more than 10⁻⁸ M, no more than 10⁻⁹ M, no more than 10⁻¹⁰ M, no more than 10⁻¹¹ M, no more than 10⁻¹² M.

In certain embodiments, the antibodies or antigen binding fragments thereof is capable of inhibiting binding of human PCSK9 to its ligand at an IC50 of no more than 10 nM, no more than 5 nM or no more than 3 nM (e.g. no more than 30 nM, 20 nM, 15 nM, 9 nM, 8 nM, 7 nM, 6 nM, 4 nM, 2 nM, or 1 nM). In certain embodiments, the antibodies or antigen binding fragments thereof is capable of binding to human PCSK9 at an EC50 of no more than 0.15 nM (e.g. no more than 1.5, 1, 0.9, 0.8, 0.7, 0.6, 0.5, 0.4, 0.3, 0.2, 0.1 or 0.05 nM).

In certain embodiments, the antibodies or antigen binding fragments thereof is capable of restoring cellular LDL uptake at an IC50 of no more than 140 nM, no more than 92 nM or no more than 80 nM (e.g. no more than 180 nM, 120 nM, 100 nM, 85 nM, 70 nM, 60 nM, 50 nM, 40 nM, or 30 nM).

In certain embodiments, the antibodies or antigen binding fragments thereof is stable in serum for at least 3 days, at least 4 days, at least 5 days, at least one week, at least two weeks, or at least one month.

In certain embodiments, the antibodies or antigen binding fragments thereof does not mediate ADCC or CDC or both.

In certain embodiments, the antibodies or antigen binding fragments thereof is a humanized monoclonal antibody. In certain embodiments, the humanized monoclonal antibody is produced by a host cell.

In certain embodiments, the antibodies or antigen binding fragments thereof is capable of reducing the level of LDL-cholesterol up to 10%, 15%, 20%, 25%, 30%, 35%, 40%, 45%, 50%, 55%, 60%, 65%, 70%, 75%, 80%, 85%, 90%, 95% or more in an animal. In certain embodiments, the antibodies or antigen binding fragments thereof is capable of maintaining the level of HDL-cholesterol.

In certain embodiments, the antibodies or antigen binding fragments thereof has a serum half-life of at least 110 hours or at least 195 hours (e.g. at least 50, at least 60, at least 70, at least 80, at least 90, at least 100, at least 150, at least 180, at least 200, at least 250, at least 300, at least 350, at least 400, at least 450, or at least 500 hours).

In one aspect, the present disclosure provides an antibody or an antigen binding fragment thereof, which competes for the same epitope with the antibody or the antigen binding fragment thereof provided herein.

In certain embodiments, the antibodies or antigen binding fragments thereof is a camelized single domain antibody, a diabody, a scFv, an scFv dimer, a BsFv, a dsFv, a (dsFv)2, a dsFv-dsFv′, an Fv fragment, a Fab, a Fab′, a F(ab′)2, a ds diabody, a nanobody, a domain antibody, or a bivalent domain antibody.

In certain embodiments, the antibodies or antigen binding fragments thereof further comprises an immunoglobulin constant region.

In certain embodiments, the antibodies or antigen binding fragments thereof further comprises a conjugate. In certain embodiments, the conjugate can be a detectable label, a pharmacokinetic modifying moiety, or a purification moiety.

In one aspect, the present disclosure further provides an isolated polynucleotide encoding the antibody or an antigen binding fragment thereof provided herein. The present disclosure further provides a vector comprising said isolated polynucleotide. The present disclosure further provides a host cell comprising said vector. In certain embodiments, the polynucleotides provided herein are operably associated with a promoter such as a SV40 promoter in a vector. In certain embodiments, host cells comprising the vectors provided herein are Chinese hamster ovary cell, or 293 cell.

In one aspect, the present disclosure further provides a method of expressing the antibody or antigen-binding fragment thereof provided herein, comprising culturing said host cell under the condition at which said polynucleotide is expressed.

In one aspect, the present disclosure further provides a kit comprising the antibody or antigen-binding fragment thereof of provided herein.

In one aspect, the present disclosure further provides a method of treating a disease or condition mediated by PCSK9 in an individual, comprising: administering a therapeutically effective amount of antibody or antigen-binding fragment thereof of provided herein to the individual. In certain embodiments, the individual has been identified as upregulated level of serum LDL cholesterol, total cholesterol and/or non-HDL cholesterol or downregulated level of LDL receptor in a test biological sample from the individual. In certain embodiments, upon administration of said antibody or antigen-binding fragment thereof, the level of LDL-C and/or total cholesterol is reduced.

In one aspect, the present disclosure further provides a pharmaceutical composition comprising the antibody or antigen-binding fragment thereof of provided herein and one or more pharmaceutically acceptable carriers. In certain of these embodiments, the pharmaceutical carriers may be, for example, diluents, antioxidants, adjuvants, excipients, or non-toxic auxiliary substances.

In one aspect, the present disclosure further provides a method of treating a condition in a subject that would benefit from upregulation of immune response, comprising administering a therapeutically effective amount of the antibody or antigen-binding fragment thereof of provided herein to the subject. In certain embodiments, the subject has upregulated level of serum LDL cholesterol, total cholesterol and/or non-HDL cholesterol or downregulated level of LDL receptor.

In one aspect, the present disclosure further provides use of the antibody or antigen-binding fragment thereof provided herein in the manufacture of a medicament for treating a condition that would benefit from upregulation of immune response. In certain embodiments, the condition is cardiovascular diseases, inflammatory diseases, and infectious diseases. In certain embodiments, the infectious disease is sepsis.

### BRIEF DESCRIPTION OF FIGURES

FIG. 1A-1C present the binding of selected anti-PCSK9 hybridoma subclones to human PCSK9 as measured by ELISA.

FIG. 2A-2C present the blocking of selected hybridoma subclones of PCSK9 antibodies to the binding of PCSK9 to LDL receptor (LDL-R) as measured by ELISA.

FIGS. 3A and 3B show the results of restoring the low-density lipoprotein (LDL)-uptake assay of selected clones of PCSK9 antibodies in liver hepatocellular carcinoma (HepG2) cell.

FIG. 4 shows the results of ELISA binding assay of selected antibodies to rhesus monkey PCSK9.

FIG. 5 shows the binding of humanized antibody 2.12.12.4-z4-uIgG4k and Repatha (also called evolocumab) to human PCSK9 as measured by ELISA binding assay.

FIG. 6 shows the blocking results of humanized antibody 2.12.12.4-z4-uIgG4k and Repatha to the binding of PCSK9 and LDL-R as measured by ELISA assay.

FIGS. 7A and 7B illustrate the results of restoring LDL-uptake assay of humanized antibodies 2.12.12.4-z1-IgG4K, 2.12.12.4-z2-IgG4K and Repatha in HepG2 and Huh-7 cells.

FIG. 8 illustrates the stability of humanized antibody 2.12.12.4-z4-uIgG4k and BMK. 115 incubated with human serum as indicated by the concentration measured by ELISA binding assay.

FIG. 9 shows staining of humanized 2.12.12.4-z4-uIgG4k in SDS-PAGE gel. M: Protein Marker; Lane1: 2.12.12.4-z4-uIgG4k, Reduced; Lane2: 2.12.12.4-z4-uIgG4k, Non-reduced.

FIG. 10 shows 100% purity of humanized 2.12.12.4-z4-uIgG4k as measured by HPLC-SEC.

FIG. 11 shows LDL-C change percentage of antibody (2.12.12.4-z4-uIgG4k or Repatha) treated cynomolgus monkeys. FIG. 11A shows the result of a single dose of 3 mg/kg injection, and FIG. 11B shows the result of a single dose of 10 mg/kg injection.

FIG. 12 shows High Density Lipoprotein cholesterol (HDL-C) change percentage of antibody (2.12.12.4-z4-uIgG4k or Repatha) treated cynomolgus monkeys. FIG. 12A shows the result of a single dose of 3 mg/kg injection, and FIG. 12B shows the result of a single dose of 10 mg/kg injection.

FIG. 13 shows antibody concentration of 2.12.12.4-z4-uIgG4k or Repatha of predose and postdose in cynomolgus monkey serum, as measured by ELISA. FIG. 13A shows the result of a single dose of 3 mg/kg injection, and FIG. 13B shows the result of a single dose of 10 mg/kg injection.

FIG. 14 shows anti-drug antibody (ADA) against 2.12.12.4-z4-uIgG4k in cynomolgus monkey serum samples of predose and postdose. FIG. 14A shows the result of a single dose of 3 mg/kg injection, and FIG. 14B shows the result of a single dose of 10 mg/kg injection.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multispecific antibody, or bispecific (bivalent) antibody that binds to a specific antigen. A native intact antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ, and mammalian light chains are classified as λ or K. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variables region in both chains generally contain three highly variable loops called the complementarity determining regions (CDRs) (light (L) chain CDRs including LCDR1, LCDR2, and LCDR3, heavy (H) chain CDRs including HCDR1, HCDR2, HCDR3). CDR boundaries for the antibodies and antigen-binding fragments disclosed herein may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding fragment is capable of binding to the same antigen to which the parent antibody binds. In certain embodiments, an antigen-binding fragment may comprise one or more CDRs from a particular human antibody grafted to a framework region from one or more different human antibodies.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab″)₂” refers to a dimer of Fab′.

“Fc” with regard to an antibody refers to that portion of the antibody consisting of the second and third constant regions of a first heavy chain bound to the second and third constant regions of a second heavy chain via disulfide bonding. The Fc portion of the antibody is responsible for various effector functions such as ADCC, and CDC, but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)). “Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

“Diabodies” include small antibody fragments with two antigen-binding sites, wherein the fragments comprise a V_{H} domain connected to a V_{L} domain in the same polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

In certain embodiments, a “(dsFv)₂” comprises three peptide chains: two V_{H} moieties linked by a peptide linker and bound by disulfide bridges to two V_{L} moieties.

In certain embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In certain embodiments, a “bispecific dsFv” or dsFv-dsFv′” comprises three peptide chains: a V_{H1}-V_{H2} moiety wherein the heavy chains are linked by a peptide linker (e.g., a long flexible linker) and bound to V_{L1} and V_{L2} moieties, respectively, via disulfide bridges, wherein each disulfide paired heavy and light chain has a different antigen specificity.

In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'S of the other moiety and form two binding sites which can target the same antigens (or eptipoes) or different antigens (or eptipoes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

The term “humanized” as used herein, with reference to antibody or antigen-binding fragment, means that the antibody or the antigen-binding fragment comprises CDRs derived from non-human animals, FR regions derived from human, and when applicable, the constant regions derived from human. A humanized antibody or antigen-binding fragment is useful as human therapeutics in certain embodiments because it has reduced immunogenicity in human. In some embodiments, the non-human animal is a mammal, for example, a mouse, a rat, a rabbit, a goat, a sheep, a guinea pig, or a hamster. In some embodiments, the humanized antibody or antigen-binding fragment is composed of substantially all human sequences except for the CDR sequences which are non-human. In some embodiments, the FR regions derived from human may comprise the same amino acid sequence as the human antibody from which it is derived, or it may comprise some amino acid changes, for example, no more than 10, 9, 8, 7, 6, 5, 4, 3, 2, or 1 changes of amino acid. In some embodiments, such change in amino acid could be present in heavy chain FR regions only, in light chain FR regions only, or in both chains. In some preferable embodiments, the humanized antibodies comprise human FR1-3 and human JH and JK.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human species, such as from mouse.

“PCSK9” as used herein refers to Proprotein Convertase Subtilisin/Kexin type 9, a naturally-occurring human proprotein convertase belonging to the proteinase K subfamily of the secretory subtilase family. PCSK9 is synthesized as a soluble zymogen that undergoes autocatalytic intramolecular processing in the endoplasmic reticulum, and is thought to function as a proprotein convertase. PCSK9 has critical role in regulating blood cholesterol levels. Gain of function mutations of PCSK9 (such as S127R, F216L, and D374Y) may associate with a form of autosomal dominant familial hypercholesterolemia, in which PCSK9 mutants enhance the level of LDL receptor. See, e.g., Burnett and Hooper, Clin Biochem Rev (2008) 29(1): 11-26, Benjannet et al. J. Biol. Chem., (2004) 279(47):48865-48875 and Fasano T et al., Atherosclerosis. (2009) 203(1):166-71. Representative amino acid sequence of human PCSK9 is disclosed under the GenBank accession number: NP_777596.2, and the representative mRNA nucleic acid sequence encoding the human PCSK9 is shown under the GenBank accession number: FJ525880.1. In certain embodiments, the term PCSK9 encompasses PCSK9 molecules of post-translational modifications of the PCSK9 amino acid sequence, such as glycosylated, PEGylated PCSK9 sequences, PCSK9 sequences with its signal sequence being cleaved, or PCSK9 sequence with its pro domain being cleaved from the catalytic domain but not separated from the catalytic domain.

“LDL-C” as used herein refers to low-density lipoprotein cholesterol and “HDL-C” refers to high-density lipoprotein cholesterol. LDL and HDL are within the five major groups of lipoprotein: chylomicrons, very low-density lipoprotein (VLDL), intermediate-density lipoprotein (IDL), low-density lipoprotein and high-density lipoprotein (HDL) (in the order from the largest particles to most dense (smallest particles). LDL (“bad” cholesterol containing particle) can transport lipid/sterol molecules, such as cholesterol (i.e. LDL-C) into artery walls, attract macrophages thus triggering atherosclerosis. In contrast, HDL (“good” cholesterol containing particle) can remove lipid molecules, such as cholesterol (i.e. HDL-C) from macrophages in the wall of arteries. Thus, high level of LDL-C has been a major risk of cardiovascular diseases (CVDs), such as peripheral artery disease, coronary artery diseases (CAD, such as angina and myocardial infarction (commonly known as a heart attack), hyperlipidemia, hypercholesterolemia, or hypertriglyceridemia), atherosclerosis, stroke, hypertensive heart disease, rheumatic heart disease, cardiomyopathy, heart arrhythmia, congenital heart disease, valvular heart disease, carditis, aortic aneurysms, peripheral artery disease, obesity, cholestatic liver disease, nephrotic syndrome, hypothyroidism and venous thrombosis, or a combination thereof.

“LDL-R” or “LDL receptor” is a mosaic cell-surface protein of 839 amino acids (after removal of 21-amino acid signal peptide) that mediates the endocytosis of LDL-C and removes LDL-C from the blood. Representative amino acid sequence of human LDL-R is disclosed under the GenBank accession number: P01130.1, and the representative mRNA nucleic acid sequence encoding the human LDL-R is shown under the GenBank accession number: NM_000527.4. When PCSK9 binds to the LDL receptor, the receptor is broken down and cannot remove LDL-C from the blood. In contrary, when PCSK9 is blocked, more LDL receptors will be present on the surface of the liver and will remove more LDL cholesterol from the blood. “Anti-PCSK9 antibody” as used herein refers to an antibody that is capable of specific binding to PCSK9 (e.g. human or monkey PCSK9) with an affinity which is sufficient to provide for diagnostic and/or therapeutic use.

The term “specific binding” or “specifically binds” as used herein refers to a non-random binding reaction between two molecules, such as for example between an antibody and an antigen. In certain embodiments, the antibodies or antigen-binding fragments provided herein specifically bind human and/or PCSK9 with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤2×10⁻⁹ M, ≤10⁻⁹ M, 10⁻¹⁰ M). KD as used herein refers to the ratio of the dissociation rate to the association rate (k_{off}/kₒₙ), may be determined using surface plasmon resonance methods for example using instrument such as Biacore.

The ability to “block binding” or “compete for the same epitope” as used herein refers to the ability of an antibody or antigen-binding fragment to inhibit the binding interaction between two molecules (e.g. human PCSK9 and an anti-PCSK9 antibody) to any detectable degree. In certain embodiments, an antibody or antigen-binding fragment that blocks binding between two molecules inhibits the binding interaction between the two molecules by at least 50%. In certain embodiments, this inhibition may be greater than 60%, greater than 70%, greater than 80%, or greater than 90%.

The term “epitope” as used herein refers to the specific group of atoms or amino acids on an antigen to which an antibody binds. Two antibodies may bind the same epitope within an antigen if they exhibit competitive binding for the antigen. For example, if an antibody or antigen-binding fragment as disclosed herein blocks binding of the exemplary antibodies such as 2.6.6, 2.12.12.4, 2.6.6-z4-uIgG1k, 2.12.12.4-z1-IgG4k, 2.12.12.4-z2-IgG4k, 2.12.12.4-z4-uIgG4k, 2.12.12.4-v2-z4-uIgG4k to human PCSK9, then the antibody or antigen-binding fragment may be considered to bind the same epitope as those exemplary antibodies.

The various symbols used in the antibody names as provided herein are of different representation: “uIgG4” refers to an antibody with human constant region of IgG4 isotype, such as uIgG1 and uIgG2 refer to antibodies with human constant regions of IgG1 and IgG2, respectively; “z” refers to a humanized antibody and z1, z2, z4 and the like refer to different versions of the humanized antibodies; “K” or “L” refers to an antibody using the kappa or lambda light chain.

“2.6.6” as used herein refers to a mouse monoclonal antibody having a heavy chain variable region of SEQ ID NO: 36, a light chain variable region of SEQ ID NO: 38. “2.6.6-z4-uIgG1k” as used herein refers to a humanized monoclonal antibody of 2.6.6 hat has a heavy chain variable region of SEQ ID NO: 48 and a light chain variable region of SEQ ID NO: 50 with a human constant region of IgG1 isotype.

“2.12.12.4” as used herein refers to a mouse monoclonal antibody having a heavy chain variable region of SEQ ID NO: 40, a light chain variable region of SEQ ID NO: 42. “2.12.12.4-z4-uIgG4k” as used herein refers to a version 4 of the humanized monoclonal antibody 2.12.12.4 with a human constant region of IgG4 isotype. 2.12.12.4-z1-IgG4K and 2.12.12.4-z2-IgG4K are humanized monoclonal antibody of 2.12.12.4 of different versions (i.e. versions 1 and 2).

“2.12.12.4-v2-z4-uIgG4k” as used herein refers to an engineered humanized antibody based on 2.12.12.4-z4-uIgG4k with mutation of two amino acids (D30E and N85D) that has a heavy chain variable region of SEQ ID NO: 44 and a light chain variable region of SEQ ID NO: 46 with a human constant region of IgG4 isotype.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and phagocytosis.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. In certain embodiments, the antibodies and antigen-binding fragments have a purity of at least 90%, 93%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

The phrase “host cell” as used herein refers to a cell into which an exogenous polynucleotide and/or a vector has been introduced.

A “disease or condition mediated by PCSK9” as used herein refers to a disease or condition caused by or characterized by a change in PCSK9, e.g. a change in expression level, in activity, and/or the presence of a variant or mutation of PCSK9. Examples of a disease or condition mediated by PCSK9 includes, but not limited to, a lipid disorder, hyperlipoproteinemia, hyperlipidemia; dyslipidemia; hypercholesterolemia, a heart attack, a stroke, coronary heart disease, atherosclerosis, peripheral vascular disease, claudication, type II diabetes, high blood pressure, a cardiovascular disease or condition, an inflammatory or autoimmune disease or condition. Methods of identification/diagnosis of above diseases or conditions are known in the art. With regard to the use of the antibodies or antigen-binding fragments disclosed herein to treat CVD (such as acute myocardial infarction (AMI), acute coronary syndrome (ACS), stroke, and CV death), a “therapeutically effective amount” as used herein refers to the dosage or concentration of the antibody or antigen-binding fragment capable of lowering lipid (such as cholesterol) in the plasma or serum, ameliorating any symptom or marker associated with CVD condition, preventing or delaying the development of a CVD condition, or some combination thereof.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

Anti-PCSK9 Antibody

In certain embodiments, the present disclosure provides exemplary humanized monoclonal antibodies 2.6.6, 2.12.12.4, 2.6.6-z4-uIgG1k, 2.12.12.4-z1-IgG4k, 2.12.12.4-z2-IgG4k, 2.12.12.4-z4-uIgG4k, 2.12.12.4-v2-z4-uIgG4k, whose CDR sequences are shown in the below Table 1, and heavy or light chain variable region sequences of the mouse parental antibody and humanized version are also shown below.

TABLE 1

| | CDR1 | CDR2 | CDR3 |
|---|---|---|---|
| 2.6.6-VH | SEQ ID NO: 1 | SEQ ID NO: 3 | SEQ ID NO: 5 |
| | SYWIH | ELNPSDGRTNYNEKF | SNFYGSSSFAN |
| | | KN | |
| | SEQ ID NO: 2 | SEQ ID NO: 4 | SEQ ID NO: 6 |
| | AGC TAC TGG ATA | GAG CTT AAT CCT | TCG AAT TTC TAC |
| | CAC | AGC GAC GGT CGT | GGT AGT AGC TCC |
| | | ACT AAC TAC AAT | TTT GCT AAC |
| | | GAG AAG TTC AAG | |
| | | AAC | |
| 2.6.6-VL | SEQ ID NO: 7 | SEQ ID NO: 9 | SEQ ID NO: 11 |
| | SASSSVSYMN | GISNLAS | QQRSSYPPT |
| | SEQ ID NO: 8 | SEQ ID NO: 10 | SEQ ID NO: 12 |
| | AGT GCC AGC TCA | GGT ATA TCC AAC | CAG CAA AGG |
| | AGT GTA AGT TAC | CTG GCT TCT | AGT AGT TAC CCA |
| | ATG AAC | | CCC ACG |
| 2.12.12.4- | SEQ ID NO: 13 | SEQ ID NO: 15 | SEQ ID NO: 17 |
| VH | NYVMH | YINPYNDYIKYNEKF | QRYYGYRPYYAM |
| | | KG | DY |
| | SEQ ID NO: 14 | SEQ ID NO: 16 | SEQ ID NO: 18 |
| | AAC TAT GTT ATG | TAT ATT AAT CCT | CAG AGG TAC TAC |
| | CAC | TAT AAT GAT TAT | GGT TAT AGA CCC |
| | | ATT AAG TAC AAT | TAC TAT GCT ATG |
| | | GAG AAG TTC AAA | GAC TAT |
| | | GGC | |
| 2.12.12.4- | SEQ ID NO: 19 | SEQ ID NO: 21 | SEQ ID NO: 23 |
| VL | RASESVDGYGNVF | LASNLES | QQNNKDPWT |
| | MH | | |
| | SEQ ID NO: 20 | SEQ ID NO: 22 | SEQ ID NO: 24 |
| | AGA GCC AGT | CTT GCA TCT AAC | CAG CAA AAT AAT |
| | GAA AGT GTT GAT | CTG GAA TCT | AAG GAT CCG TGG |
| | GGT TAT GGC AAT | | ACG |
| | GTT TTT ATG CAC | | |
| 2.12.12.4- | SEQ ID NO: 13 | SEQ ID NO: 15 | SEQ ID NO: 17 |
| v2-z4-VH | NYVMH | YINPYNDYIKYNEKF | QRYYGYRPYYAM |
| | | KG | DY |
| | SEQ ID NO: 27 | SEQ ID NO: 28 | SEQ ID NO: 29 |
| | AAC TAC GTG ATG | TAT ATT AAC CCC | CAG CGG TAC TAC |
| | CAC | TAC AAC GAC TAC | GGC TAC AGG CCC |
| | | ATC AAG TAC AAT | TAC TAC GCC ATG |
| | | GAG AAG TTT AAA | GAT TAT |
| | | GGG | |
| 2.12.12.4- | SEQ ID NO: 25 | SEQ ID NO: 21 | SEQ ID NO: 23 |
| v2-z4-VL | RASESVEGYGNVF | LASNLES | QQNNKDPWT |
| | MH | | |
| | SEQ ID NO: 26 | SEQ ID NO: 22 | SEQ ID NO: 24 |
| | CGG GCC TCA GAA | CTG GCC AGC AAC | CAG CAG AAC |
| | AGT GTG GAG | CTC GAG AGC | AAC AAG GAC CCC |
| | GGA TAC GGG | | TGG ACA |
| | AAC GTG TTC ATG | | |
| | CAC | | |
| 2.6.6-z4- | SEQ ID NO: 1 | SEQ ID NO: 3 | SEQ ID NO: 5 |
| VH | SYWIH | ELNPSDGRTNYNEKF | SNFYGSSSFAN |
| | | KN | |
| | SEQ ID NO: 30 | SEQ ID NO: 31 | SEQ ID NO: 32 |
| | AGC TAC TGG ATC | GAG CTG AAC CCC | TCA AAC TTC TAT |
| | CAC | AGT GAT GGG AGG | GGC AGC AGT AGC |
| | | ACA AAT TAC AAC | TTT GCC AAC |
| | | GAG AAG TTT AAG | |
| | | AAC | |
| 2.6.6-z4- | SEQ ID NO: 7 | SEQ ID NO: 9 | SEQ ID NO: 11 |
| VL | SASSSVSYMN | GISNLAS | QQRSSYPPT |
| | SEQ ID NO: 33 | SEQ ID NO: 34 | SEQ ID NO: 35 |
| | TCC GCC AGT TCA | GGA ATC TCC AAC | CAG CAG AGG |
| | TCA GTG AGT TAC | CTT GCC AGC | AGC AGC TAT CCA |
| | ATG AAT | | CCC ACC |

2.6.6-VH (mouse antibody)
Amino acid sequence (SEQ ID NO: 36):
QVQLQQPGAEVVKPGASVTVSCKASGYTFTSYWIHWVMQRPGQGLEWIGELNPSD
GRTNYNEKFKNKATLTVDTSSSTVYMQLSSLTSEDSAVYYCARSNFYGSSSFANWG
QGTLVTVSA
Nucleic acid sequence (SEQ ID NO: 37)
CAGGTCCAACTGCAGCAGCCTGGGGCTGAAGTGGTGAAGCCTGGGGCTTCAGTG
ACGGTGTCCTGCAAGGCTTCTGGCTACACCTTCACCAGCTACTGGATACACTGGG
TGATGCAGAGACCTGGACAAGGCCTTGAGTGGATTGGAGAGCTTAATCCTAGCG
ACGGTCGTACTAACTACAATGAGAAGTTCAAGAACAAGGCCACACTGACTGTAG
ACACATCGTCCAGTACAGTCTACATGCAACTCAGCAGCCTGACATCTGAGGACTC
TGCGGTCTATTACTGTGCAAGATCGAATTTCTACGGTAGTAGCTCCTTTGCTAACT
GGGGCCAAGGGACTCTGGTCACTGTCTCTGCA
2.6.6 - VL (mouse antibody)
Amino acid sequence (SEQ ID NO: 38):
EILLTQSPAIIAASPGEKVTITCSASSSVSYMNWYQQKPGSSPILWIYGISNLASGVPAR
FSGSGSGTSFSFTINNMEAEDVATYYCQQRSSYPPTFGGGTKLEIK
Nucleic acid sequence (SEQ ID NO: 39)
GAAATTTTGCTCACCCAGTCTCCAGCAATCATAGCTGCATCTCCTGGGGAGAAGG
TCACCATCACCTGCAGTGCCAGCTCAAGTGTAAGTTACATGAACTGGTATCAGCA
GAAACCAGGATCCTCCCCCATACTATGGATCTATGGTATATCCAACCTGGCTTCT
GGAGTTCCTGCTCGCTTCAGCGGCAGTGGGTCTGGGACATCTTTCTCTTTCACAAT
CAACAACATGGAGGCTGAAGATGTTGCCACTTATTACTGTCAGCAAAGGAGTAG
TTACCCACCCACGTTCGGAGGGGGGACCAAGCTGGAAATAAAA
2.12.12.4-VH (mouse antibody)
Amino acid sequence (SEQ ID NO: 40):
EVQLQQSGPELVKPGASVKMSCEASGYTFTNYVMHWVKQKPGQGLEWIGYINPYN
DYIKYNEKFKGKATLTSDKSSSTTYLEVSSLTSEDSAVYYCGRQRYYGYRPYYAMD
YWGQGTSVTVSS
Nucleic acid sequence (SEQ ID NO: 41)
GAGGTCCAGCTGCAGCAGTCTGGACCTGAGCTGGTAAAGCCTGGGGCCTCAGTG
AAGATGTCCTGCGAGGCTTCTGGATACACATTCACTAACTATGTTATGCACTGGG
TGAAGCAGAAGCCTGGGCAGGGCCTTGAGTGGATTGGATATATTAATCCTTATAA
TGATTATATTAAGTACAATGAGAAGTTCAAAGGCAAGGCCACACTGACTTCAGA
CAAATCCTCCAGTACAACCTACCTGGAAGTCAGCAGCCTGACCTCTGAGGACTCT
GCGGTCTATTACTGTGGAAGACAGAGGTACTACGGTTATAGACCCTACTATGCTA
TGGACTATTGGGGTCAGGGAACCTCAGTCACCGTCTCCTCA
2.12.12.4-VL (mouse antibody)
Amino acid sequence (SEQ ID NO: 42):
NIVLTQSPASLAVSLGQRATISCRASESVDGYGNVFMHWYQQKPGQPPKLLIYLASN
LESGVPDRFSGSGSRTDFTLTIDPVEADDAATYYCQQNNKDPWTFGGGTKLEIK
Nucleic acid sequence (SEQ ID NO: 43)
AACATTGTGCTGACCCAATCTCCAGCTTCTTTGGCTGTGTCTCTAGGTCAGAGGG
CCACCATTTCCTGCAGAGCCAGTGAAAGTGTTGATGGTTATGGCAATGTTTTTAT
GCACTGGTACCAGCAGAAACCAGGACAGCCACCCAAACTCCTCATCTATCTTGCA
TCTAACCTGGAATCTGGGGTCCCTGACAGGTTCAGTGGCAGTGGGTCTAGGACAG
ACTTCACCCTCACCATTGATCCTGTGGAGGCTGATGATGCTGCAACTTATTACTGT
CAGCAAAATAATAAGGATCCGTGGACGTTCGGTGGAGGCACCAAGCTGGAAATCAAA
2.12.12.4-v2-Z4-uIgG4k-VH
(engineered humanized antibody, D30E and N85D)
Amino acid sequence (SEQ ID NO: 44):
QVQLVQSGAEVKKPGSSVKVSCEASGYTFTNYVMHWVRQAPGQGLEWMGYINPY
NDYIKYNEKFKGKATITADKSTSTAYMELSSLRSEDTAVYYCGRQRYYGYRPYYAM
DYWGQGTLVTVSS
Nucleic acid sequence (SEQ ID NO: 45)
CAAGTGCAGCTCGTGCAGTCTGGAGCAGAAGTGAAGAAGCCTGGATCATCCGTC
AAGGTGAGTTGCGAGGCTTCCGGGTACACATTCACCAACTACGTGATGCACTGG
GTCAGACAGGCTCCAGGGCAGGGGCTTGAGTGGATGGGGTATATTAACCCCTAC
AACGACTACATCAAGTACAATGAGAAGTTTAAAGGGAAAGCCACCATCACTGCC
GACAAGAGCACCAGCACAGCCTACATGGAGCTGTCCAGCCTGAGGAGCGAGGAT
ACAGCCGTCTACTACTGTGGCCGCCAGCGGTACTACGGCTACAGGCCCTACTACG
CCATGGATTATTGGGGCCAGGGCACTTTGGTGACAGTCAGCTCC
2.12.12.4-v2-z4-uIgG4k-VL
(engineered humanized antibody, D30E and N85D)
Amino acid sequence (SEQ ID NO: 46):
DIVLTQSPASLAVSPGQRATITCRASESVEGYGNVFMHWYQQKPGQPPKLLIYLASN
LESGVPARFSGSGSGTDFTLTINPVEADDTANYYCQQNNKDPWTFGGGTKVEIK
Nucleic acid sequence (SEQ ID NO: 47)
GACATCGTCTTGACCCAGTCTCCTGCTTCCCTTGCAGTGTCCCCTGGGCAAAGAG
CTACTATTACCTGCCGGGCCTCAGAAAGTGTGGAGGGATACGGGAACGTGTTCAT
GCACTGGTACCAGCAGAAGCCCGGACAGCCACCAAAGCTGCTCATCTACCTGGC
CAGCAACCTCGAGAGCGGCGTGCCCGCCAGGTTTAGCGGGAGCGGCTCCGGGAC
TGATTTCACACTGACAATTAATCCCGTCGAGGCCGACGATACAGCCAACTATTAC
TGTCAGCAGAACAACAAGGACCCCTGGACATTTGGCGGCGGGACCAAAGTCGAG
ATCAAG
2.6.6-v2-z4-uIgG1k-VH
Amino acid sequence (SEQ ID NO: 48):
QVQLVQSGAEVKKPGASVKVSCKASGYTFTSYWIHWVRQAPGQGLEWMGELNPSD
GRTNYNEKFKNKATMTVDTSTSTVYMELSSLRSEDTAVYYCARSNFYGSSSFANWG
QGTLVTVSS
Nucleic acid sequence (SEQ ID NO: 49)
CAGGTGCAACTTGTGCAGTCCGGAGCAGAAGTGAAGAAGCCAGGGGCTTCAGTG
AAGGTCTCCTGCAAGGCTTCCGGATACACCTTCACCAGCTACTGGATCCACTGGG
TCAGACAGGCCCCTGGGCAGGGCTTGGAGTGGATGGGCGAGCTGAACCCCAGTG
ATGGGAGGACAAATTACAACGAGAAGTTTAAGAACAAAGCCACCATGACCGTCG
ACACATCCACAAGCACAGTGTACATGGAGCTCTCCAGCCTGCGCAGCGAGGACA
CTGCCGTCTACTACTGTGCCCGGTCAAACTTCTATGGCAGCAGTAGCTTTGCCAA
CTGGGGCCAGGGGACTCTGGTGACTGTGTCTTCT
2.6.6-v2-z4-uIgG1k-VL
Amino acid sequence (SEQ ID NO: 50):
DIQMTQSPSSLSASVGDRVTITCSASSSVSYMNWYQQKPGKAPKLLIYGISNLASGVP
SRFSGSGSGTDFTFTISSLQPEDIATYYCQQRSSYPPTFGGGTKVEIK
Nucleic acid sequence (SEQ ID NO: 51)
GACATCCAAATGACCCAGAGCCCTTCTTCCCTCTCCGCTTCTGTCGGAGATCGGG
TCACAATCACATGCTCCGCCAGTTCATCAGTGAGTTACATGAATTGGTACCAGCA
GAAGCCTGGGAAGGCACCCAAGCTGCTGATCTACGGAATCTCCAACCTTGCCAG
CGGCGTGCCAAGCAGATTCTCCGGCAGCGGGAGCGGGACAGATTTCACCTTTAC
AATTAGCTCCTTGCAGCCCGAAGACATTGCCACTTACTACTGTCAGCAGAGGAGC
AGCTATCCACCCACCTTTGGCGGCGGGACTAAAGTGGAGATTAAG

In certain embodiments, one or more CDR sequences provided herein can be modified or changed such that the resulting antibody is improved over the parent antibody in one or more properties (such as improved antigen-binding, improved glycosylation pattern, reduced risk of glycosylation on a CDR residue, reduced deamination on a CDR residue, increased pharmacokinetic half-life, pH sensitivity, and compatibility to conjugation), and is otherwise comparable to the parent antibody (i.e. antibody having otherwise the same set of CDR sequences except for the above-mentioned modification or change), or at least substantially retains the antigen-binding property of the parent antibody.

A skilled artisan will understand that the CDR sequences provided in Table 1 can be modified to contain one or more substitutions of amino acids, so as to provide for an improved biological activity such as improved binding affinity to human PCSK9. For example, a library of antibody variants (such as Fab or scFv variants) can be generated and expressed with phage display technology, and then screened for the binding affinity to human PCSK9. For another example, computer software can be used to virtually simulate the binding of the antibodies to human PCSK9, and identify the amino acid residues on the antibodies which form the binding interface. Such residues may be either avoided in the substitution so as to prevent reduction in binding affinity, or targeted for substitution to provide for a stronger binding. In certain embodiments, at least one (or all) of the substitution(s) in the CDR sequences is conservative substitution.

In certain embodiments, the antibodies and the antigen-binding fragments thereof comprise one or more CDR sequences having at least 80% (e.g. at least 85%, 88%, 90%, 91%, 92%, 93%, 94%, 95%, 96%, 97%, 98%, 99%) sequence identity to that (or those) listed in Table 1, and in the meantime retain the binding affinity to human PCSK9 at a level similar to or even higher than its parental antibody having substantially the same sequence except that the corresponding CDR sequence is in 100% sequence identity to that (or those) listed in Table 1.

In certain embodiments, the anti-PCSK9 antibodies and the antigen-binding fragments thereof are humanized. Theses humanized antibodies retain the binding affinity to human PCSK9, preferably at a level similar to one of the exemplary antibodies: 2.6.6, 2.12.12.4, 2.6.6-z4-uIgG1k, 2.12.12.4-z1-IgG4k, 2.12.12.4-z2-IgG4k, 2.12.12.4-z4-uIgG4k, 2.12.12.4-v2-z4-uIgG4k.

Also contemplated herein are antibodies and the antigen-binding fragments that compete for the same epitope with the anti-PCSK9 antibodies and the antigen-binding fragments thereof provided herein. In certain embodiments, the antibodies block binding of 2.6.6, 2.12.12.4, 2.6.6-z4-uIgG1k, 2.12.12.4-z1-IgG4k, 2.12.12.4-z2-IgG4k, 2.12.12.4-z4-uIgG4k, 2.12.12.4-v2-z4-uIgG4k to human or monkey PCSK9, for example, at an IC50 value (i.e. 50% inhibition concentration) of below 10⁻⁶ M, below 10⁻⁷ M, below 10^{-7.5} M, below 10⁻⁸ M, below 10^{-8.5} M, below 10⁻⁹ M, or below 10⁻¹⁰ M, below 10⁻¹¹ M or below 10⁻¹² M. The IC₅₀ values are determined based on a competition assay such as ELISA assays and radioligand competition binding assays.

In some embodiments, the anti-PCSK9 antibodies and the antigen-binding fragments thereof provided herein are capable of specifically binding to human PCSK9 with a binding affinity (Kd) of no more than 10⁻⁸ M, no more than 10⁻⁹ M or no more than 10⁻¹⁰ M (e.g., ≤1.1×10⁻⁹ M, ≤2×10⁻⁹ M, ≤10⁻⁹ M, ≤5.5×10⁻¹⁰ M, ≤4.5×10⁻¹¹ M, ≤5.5×10⁻¹¹ M) as measured by surface plasmon resonance binding assay or ELISA. The binding affinity can be represented by K_{D} value, which is calculated as the ratio of dissociation rate to association rate (k_{off}/kₒₙ) when the binding between the antigen and the antigen-binding molecule reaches equilibrium. The antigen-binding affinity (e.g. K_{D}) can be appropriately determined using suitable methods known in the art, including, for example, surface plasmon resonance binding assay using instruments such as Biacore (see, for example, Murphy, M. et al, Current protocols in protein science, Chapter 19, unit 19.14, 2006).

In certain embodiments, the antibodies and the fragments thereof provided herein binds to human PCSK9 with an EC₅₀ (i.e. 50% binding concentration) of 0.05 nM-1 nM (e.g. 0.1 nM-0.9 nM, 0.1 nM-0.8 nM, 0.1 nM-0.7 nM, 0.1 nM-0.6 nM, 0.1 nM-0.5 nM, 0.1 nM-0.4 nM, 0.1 nM-0.3 nM, or 0.1 nM-0.2 nM). Binding of the antibodies to human PCSK9 can be measured by methods known in the art, for example, sandwich assay such as ELISA, Western Blot, other binding assay. In an illustrative example, the test antibody (i.e. first antibody) is allowed to bind to immobilized human PCSK9, after washing away the unbound antibody, a labeled secondary antibody is introduced which can bind to and thus allow detection of the bound first antibody. The detection can be conducted with a microplate reader when immobilized PCSK9 is used.

In certain embodiments, the antibodies and the fragments thereof provided herein inhibit the binding of human PCSK9 to human LDL receptor at an IC₅₀ of 3 nM-10 nM (e.g. 3.5 nM-9.5 nM, 3.5 nM-8.5 nM, or 5 nM-8.5 nM), as measured in a competition assay.

In certain embodiments, the antibodies and antigen-binding fragments thereof bind to monkey PCSK9 with a binding affinity similar to that of human PCSK9. For example, binding of the exemplary antibodies 2.6.6, 2.12.12.4, 2.6.6-z4-uIgG1k, 2.12.12.4-z1-IgG4k, 2.12.12.4-z2-IgG4k, 2.12.12.4-z4-uIgG4k, 2.12.12.4-v2-z4-uIgG4k to monkey PCSK9 is at a similar affinity or EC50 value to that of human PCSK9.

In some embodiments, the anti-PCSK9 antibodies and the antigen-binding fragments thereof further comprise an immunoglobulin constant region. In some embodiments, an immunoglobulin constant region comprises a heavy chain and/or a light chain constant region. The heavy chain constant region comprises CH1, CH1-CH2, or CH1-CH3 regions. In some embodiments, the constant region may further comprise one or more modifications to confer desirable properties. For example, the constant region may be modified to reduce or deplete one or more effector functions, to improve FcRn receptor binding, or to introduce one or more cysteine residues. In some embodiments, the anti-PCSK9 antibodies and the antigen-binding fragments thereof have a constant region of IgG4 isotype, which has reduced or depleted effector function. Various assays are known to evaluate ADCC or CDC activities, for example, Fc receptor binding assay, C1q binding assay, and cell lysis assay, and can be readily selected by people in the art.

In certain embodiments, the antibodies and antigen-binding fragments thereof can be used as the base of antibody-drug conjugates, bispecific or multivalent antibodies.

The anti-PCSK9 antibodies or antigen-binding fragments thereof provided herein can be a monoclonal antibody, polyclonal antibody, humanized antibody, chimeric antibody, recombinant antibody, bispecific antibody, labeled antibody, bivalent antibody, or anti-idiotypic antibody. A recombinant antibody is an antibody prepared in vitro using recombinant methods rather than in animals. A bispecific or bivalent antibody is an artificial antibody having fragments of two different monoclonal antibodies and can bind two different antigens. An antibody or antigen-binding fragment thereof that is “bivalent” comprises two antigen-binding sites. The two antigen binding sites may bind to the same antigen, or they may each bind to a different antigen, in which case the antibody or antigen-binding fragment is characterized as “bispecific.”

In some embodiments, the anti-PCSK9 antibodies and the antigen-binding fragments thereof is a camelized single domain antibody, a diabody, a scFv, an scFv dimer, a BsFv, a dsFv, a (dsFv)2, a dsFv-dsFv′, an Fv fragment, a Fab, a Fab′, a F(ab′)2, a ds diabody, a nanobody, a domain antibody, or a bivalent domain antibody.

In some embodiments, the anti-PCSK9 antibodies and the antigen-binding fragments thereof further comprise a conjugate. It is contemplated that a variety of conjugates may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the antibodies or antigen-binding fragments by covalent binding, affinity binding, intercalation, coordinate binding, complexation, association, blending, or addition, among other methods. In certain embodiments, the antibodies and antigen-binding fragments disclosed herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate. In certain embodiments, the antibodies may be linked to a conjugate indirectly, or through another conjugate. For example, the antibody or antigen-binding fragments may be conjugated to biotin, then indirectly conjugated to a second conjugate that is conjugated to avidin. The conjugate can be a detectable label, a pharmacokinetic modifying moiety, a purification moiety, or a cytotoxic moiety. Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides, luminescent labels), chromophoric moiety, digoxigenin, biotin/avidin, a DNA molecule or gold for detection. In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like. In certain embodiments, the conjugate can be a purification moiety such as a magnetic bead. A “cytotoxic moiety” can be any agent that is detrimental to cells or that can damage or kill cells. Examples of cytotoxic moiety include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Polynucleotides and Recombinant Methods

The present disclosure provides isolated polynucleotides that encode the anti-PCSK9 antibodies and the antigen-binding fragments thereof. In certain embodiments, the isolated polynucleotides comprise one or more nucleotide sequences as shown in Table 1, which encodes the CDR sequences provided in Table 1.

In some embodiments, the isolated polynucleotides encodes a heavy chain variable region and comprise a sequence selected from the group consisting of: SEQ ID NO: 26, SEQ ID NO: 30, SEQ ID NO: 34, and a homologous sequence thereof having at least 80% (e.g. at least 85%, 88%, 90%, 91%, 92%, 93%, 94%, 95%, 96%, 97%, 98%, 99%) sequence identity. In some embodiments, the isolated polynucleotides encodes a light chain variable region and comprise a sequence selected from the group consisting of: SEQ ID NO: 28, SEQ ID NO: 32, SEQ ID NO: 36, and a homologous sequence thereof having at least 80% (e.g. at least 85%, 88%, 90%, 91%, 92%, 93%, 94%, 95%, 96%, 97%, 98%, 99%) sequence identity. In certain embodiments, the percentage identity is due to genetic code degeneracy, while the encoded protein sequence remains unchanged.

The isolated polynucleotide that encodes the anti-PCSK9 antibodies and the antigen-binding fragments thereof (e.g. including the sequences in Table 1) can be inserted into a vector for further cloning (amplification of the DNA) or for expression, using recombinant techniques known in the art. In another embodiment, the antibody may be produced by homologous recombination known in the art. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

In some embodiments, the vector system includes mammalian, bacterial, yeast systems, etc, and comprises plasmids such as, but not limited to, pALTER, pBAD, pcDNA, pCal, pL, pET, pGEMEX, pGEX, pCI, pCMV, pEGFP, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS420, pLexA, pACT2.2 etc, and other laboratorial and commercially available vectors. Suitable vectors may include, plasmid, or viral vectors (e.g., replication defective retroviruses, adenoviruses and adeno-associated viruses).

Vectors comprising the polynucleotide sequence encoding the antibody or antigen-binding fragment can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-PCSK9 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some preferable embodiments, the host cell is 293F cell.

Host cells are transformed with the above-described expression or cloning vectors for anti-PCSK9 antibody production and cultured in conventional nutrient media modified as appropriate for inducing promoters, selecting transformants, or amplifying the genes encoding the desired sequences.

The host cells used to produce the antibodies or antigen-binding fragments provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human .gamma.1, .gamma.2, or .gamma.4 heavy chains (Lindmark et al., J. Immunol. Meth. 62:1-13 (1983)). Protein G is recommended for all mouse isotypes and for human .gamma.3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

Kits

The present disclosure provides kits comprising the anti-PCSK9 antibodies or the antigen-binding fragments thereof. In some embodiments, the kits are useful for detecting the presence or level of PCSK9 in a biological sample. The biological sample can comprise serum. In some embodiments, the kit comprises an anti-PCSK9 antibody or the antigen-binding fragment thereof which is conjugated with a detectable label. In certain other embodiments, the kit comprises an unlabeled anti-PCSK9 antibody or antigen-binding fragment, and further comprises a secondary labeled antibody which is capable of binding to the unlabeled anti-PCSK9 antibody. The kit may further comprise an instruction of use, and a package that separates each of the components in the kit.

In some embodiments, the kits are useful for treating, preventing, or delaying diseases or conditions mediated by PCSK9. In certain embodiments, the anti-PCSK9 antibody or the antigen-binding fragment thereof are associated with a substrate or a device useful in a sandwich assay such as ELISA, or in an immunographic assay. Useful substrate or device can be, for example, microtiter plate and test strip.

In certain embodiments, the kit further comprises one or more agents known to be beneficial for reducing cholesterol. Exemplary agents include statin, an HMG-CoA reductase inhibitor other than a statin, niacin (nicotinic acid), a cholesterol absorption inhibitor, a cholesteryl ester transfer protein (CETP), a bile acid sequestrant, a fibrate, a phytosterol; or a modulator of lipid/lipid concentration ratios selected from a small molecule, peptidomimetic, an antisense RNA, a small interfering RNA (siRNA), and a natural or modified lipid. In certain embodiments, a cholesterol absorption inhibitor is ezetimibe or SCH-48461; a CETP is evacetrapib, anacetrapib or dalcetrapib; a bile acid sequestrant is preferably colesevelam, cholestyramine or colestipol; a fibrate is preferably fenofibrate, gemfibrozil, clofibrate, or bezafibrate; or the combination thereof.

Pharmaceutical Composition and Method of Treatment

The present disclosure further provides pharmaceutical compositions comprising the anti-PCSK9 antibodies or the antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-PCSK9 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

Therapeutic methods are also provided, comprising: administering a therapeutically effective amount of the antibody or antigen-binding fragment as provided herein to a subject in need thereof, thereby treating or preventing a condition or a disorder associated with related to PCSK9. In another aspect, methods are provided to treat a condition in a subject that would benefit from upregulation of immune response, comprising administering a therapeutically effective amount of the antibody or antigen-binding fragment as provided herein to a subject in need thereof.

The therapeutically effective amount of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of tumor development. Dosages may be proportionally reduced or increased by one of ordinary skill in the art (e.g., physician or veterinarian) as indicated by these and other circumstances or requirements.

In certain embodiments, an antibody or antigen-binding fragment as provided herein may be administered at a therapeutically effective dosage of about 0.01 mg/kg to about 100 mg/kg (e.g., about 0.01 mg/kg, about 0.5 mg/kg, about 1 mg/kg, about 2 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg). In certain of these embodiments, the antibody or antigen-binding fragment is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies and antigen-binding fragments disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

### METHODS OF USE

The present disclosure further provides methods of using the anti-PCSK9 antibodies or the antigen-binding fragments thereof.

In some embodiments, the present disclosure provides methods of treating a condition or a disorder associated with or mediated by PCSK9 in an individual, comprising administering a therapeutically effective amount of the anti-PCSK9 antibody or antigen-binding fragment thereof. In certain embodiments, the individual has been identified as having a disorder or condition likely to respond to a PCSK9 inhibitor. In certain embodiments, the individual is at risk of having or developing a disease or condition mediated by PCSK9 that exhibits one or more symptoms of said disease or condition, such as being overweight, having elevated cholesterol level, having genetic mutation in the genes encoding LDL-R or APOB, or having family history of such a disease or condition. In certain embodiments, the individual is resistant to or intolerable to another cholesterol lowering agents in a therapy, for example, statin, such that the level of cholesterol cannot be effectively lowered to an acceptable level in such therapy. In certain embodiments, the diseases or conditions mediated by PCSK9 include infectious disease such as severe cellulitis, gastroenteritis, sepsis, pneumonia, skin and soft tissue infections, pyelonephritis, viral infection, for example, viral infection of hepatitis B, hepatitis C, herpes virus, Epstein-Barr virus, HIV, cytomegalovirus, herpes simplex virus type I, herpes simplex virus type 2, human papilloma virus, adenovirus, Kaposi West sarcoma associated herpes virus epidemics, thin ring virus (Torquetenovirus), JC virus or BK virus, or include inflammatory diseases, such as Alzheimer's, ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease and ulcerative colitis.

The presence or level of LDL-C on an interested biological sample can be indicative of whether the individual from whom the biological sample is derived could likely respond to a PCSK9 inhibitor. Various methods can be used to determine the presence or level of LDL-C in a test biological sample from the individual. Milligrams (mg) per deciliter (dL) of blood of cholesterol levels are measured in the USA, while millimoles (mmol) per liter (L) of blood are used in Canada and many European countries.

In certain embodiments, presence or upregulated level of the LDL-C, total cholesterol or non-HDL-C in the test biological sample indicates likelihood of responsiveness. The term “upregulated” as used herein, refers to an overall increase of no less than 10%, 15%, 20%, 25%, 30%, 35%, 40%, 45%, 50%, 55%, 60%, 65%, 70%, 75%, 80% or greater, in the cholesterol level in the test sample as detected using the antibodies or antigen-binding fragments provided herein, as compared to the cholesterol level in a reference sample as detected using the same antibody. The reference sample can be a control sample obtained from a healthy or non-diseased individual, or a healthy or non-diseased sample obtained from the same individual from whom the test sample is obtained.

The antibodies or antigen-binding fragments disclosed herein may be administered alone or in combination with one or more additional therapeutic means or agents. For example, the antibodies or antigen-binding fragments disclosed herein may be administered in combination with statin, an HMG-CoA reductase inhibitor other than a statin, niacin (nicotinic acid), a cholesterol absorption inhibitor, a cholesteryl ester transfer protein (CETP), a bile acid sequestrant, a fibrate, a phytosterol; or a modulator of lipid/lipid concentration ratios selected from a small molecule, peptdomimetic, an antisense RNA, a small interfering RNA (siRNA), and a natural or modified lipid. In certain embodiments, a cholesterol absorption inhibitor is ezetimibe or SCH-48461; a CETP is evacetrapib, anacetrapib or dalcetrapib; a bile acid sequestrant is preferably colesevelam, cholestyramine or colestipol; a fibrate is preferably fenofibrate, gemfibrozil, clofibrate, or bezafibrate.

In certain of these embodiments, an antibody or antigen-binding fragment as disclosed herein that is administered in combination with one or more above additional therapeutic agents may be administered simultaneously with the one or more additional therapeutic agents, and in certain of these embodiments the antibody or antigen-binding fragment and the additional therapeutic agent(s) may be administered as part of the same pharmaceutical composition. However, an antibody or antigen-binding fragment administered “in combination” with another therapeutic agent does not have to be administered simultaneously with or in the same composition as the agent. An antibody or antigen-binding fragment administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: Antibody and Other Proteins Generation

1.1 Human and Murine PCSK9

Human and murine PCSK9 genes were inserted into expression vector pcDNA 3.3 with a 6-His tag or murine Fc (mFc) fused at the C-terminus. The plasmids were then transfected to HEK293 cells using PlasFect (Bioline USA, BIO-46026). The His-tag proteins were purified from harvested supernatant using a Ni column (Qiagen Inc). The mFc-fused proteins were purified using Protein A column (MabSelect SuRe, GE).

1.2 Human LDL-R

The gene of LDL receptor extracellular domain was inserted into vector pcDNA 3.3 with a C-terminal 6-His tag. The plasmid was transfected to HEK293 cells using PlasFect (Bioline USA, BIO-46026). LDL-R protein was firstly purified from harvested supernatant using a Ni column (Qiagen Inc), followed by purification using ion-exchange column.

1.3 Reference Antibody

Reference antibody BMK.115 was generated based on the sequence of 21B12 in U.S. Pat. No. 8,889,834B2. The plasmids containing VH and VL gene were co-transfected into HEK293 cells. Antibody was purified form harvested supernatant using Protein A column (MabSelect SuRe, GE).

### Example 2: Antibody Generation

2.1 Immunization

Balb/c mice were injected with human PCSK9 protein via foot pads approximately every 3 days. First titer test was performed after 6 times injection. Afterwards, the rats were injected every other week.

2.2 Serum Titer Detection

ELISA assay was used to measure titers of antibody in mouse serum. ELISA plates (Nunc) were coated with human PCSK9 at 1 μg/ml overnight at 4° C., and then blocked with blocking buffer (1×PBS/2% BSA) for 1 h at room temperature. Mouse serum was 1:3 titrated starting at 1:100 dilution in blocking buffer and incubated for 1 h at room temperature. The plates were then washed and subsequently incubated with secondary antibody goat anti mouse-IgG-Fc-HRP (Bethyl) for 1 h. After washing, TMB substrate was added and the interaction was stopped by 2M HCl. The absorbance at 450 nm was read using a microplate reader (Molecular Device).

2.2.1 Immunization and Hybridoma Generation

Titers of the antigen-specific antibody in serum were determined by ELISA assay (Table 2). All the mice showed PCSK9 specific titer. The mice with serum titer of 656100 or higher were selected for hybridoma fusion.

TABLE 2
Summary of serum titer

| Mouse# | 1 | 2 | 3 | 4 | 5 |
|---|---|---|---|---|---|
| Pre-bleed | <100 | <100 | <100 | <100 | <100 |
| 1st-bleed | 218700 | 218700 | 656100 | 218700 | 656100 |
| 2^{nd}-bleed | 218700 | 656100 | 656100 | 218700 | 656100 |

2.3 Hybridoma Generation

Lymph nodes and spleen were collected from immunized mice under sterile condition, and lymphocytes were prepared using Ficoll-Paque PLUS gradient centrifugation. The isolated cells were then fused with myeloma cell SP2/0 at a ratio of 1:1 using electrofusion device (BTX ECM2001). Cells were transferred to 2 HA media after fusion. 5×10⁵ cells were seeded per 96-well plate.

2.4 Hybridoma Screening

Binding assay by ELISA: Plates (Nunc) were coated with Streptavidin at 1 μg/ml overnight at 4° C. After blocking and washing, 250 ng/ml PCSK9-His-biotin was added and incubated for 1 h. Then hybridoma supernatants were transferred to the plates and incubate at room temperature for 1 h. The plates were then washed and subsequently incubated with secondary antibody goat anti mouse-IgG-Fc-HRP (Bethyl) for 1 h. After washing, TMB substrate was added and the interaction was stopped by 2M HCl. The absorbance at 450 nm was read using a microplate reader (Molecular Device).

Blocking assay by ELISA: Plates (Nunc) were coated with LDL-R overnight at 4° C. Hybridoma supernatants were mixed with 250 ng/ml PCSK9-mFc-biotin and incubated at 4° C. overnight. After blocking and washing, the mixture were added to the plates and incubated for 1 h. The plates were then washed and subsequently incubated with streptavidin-HRP. After washing, TMB substrate was added and the interaction was stopped by 2M HCl. The absorbance at 450 nm was read using a microplate reader (Molecular Device).

Antibody Screening

Hybridoma supernatant was used for primary screen. The primary binding screen identified a total of 1090 hybridomas which can produce antigen-specific binding antibodies. The antigen-specific hybridomas were then screened by ELISA blocking assay. The blocking assay resulted in 54 hybridomas which can block the binding of human PCSK9 to human LDL-R. 17 selected hybridoma lines were subcloned by limiting dilution. Hybridoma subclones were verified by binding and blocking ELISA assay, and their isotypes were also detected.

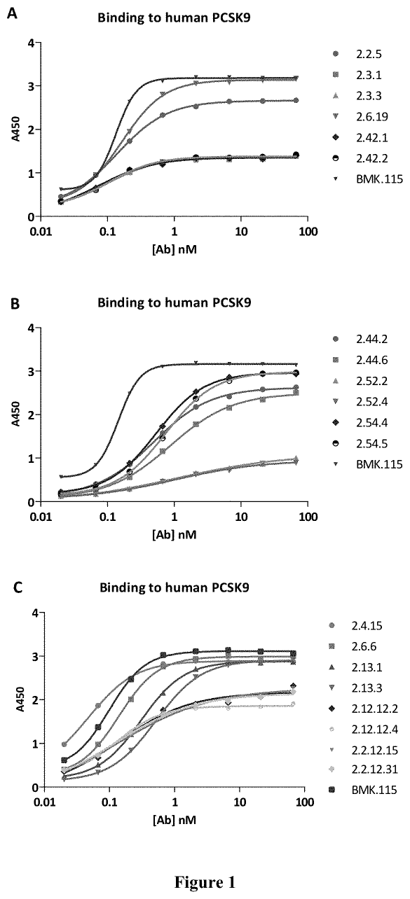

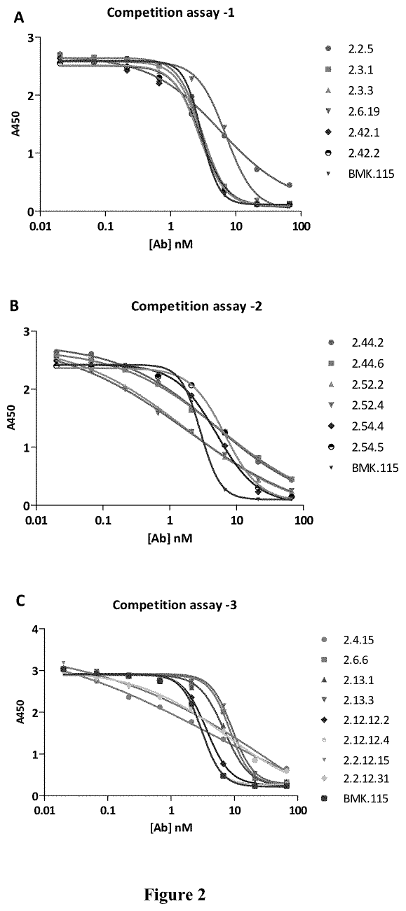

Binding and blocking activities were confirmed using purified antibodies (FIGS. 1 and 2). The binding EC50 and blocking IC50 values were summarized in Table 3.

TABLE 3
Summary of binding and blocking activities
of selected hybridoma clones

| | Binding | Competition |
|---|---|---|
| Antibody | EC50 (nM) | IC50 (nM) |
| 2.4.15 | 0.05 | NA |
| 2.6.6 | 0.14 | 8.5 |
| 2.13.1 | 0.31 | 6.9 |
| 2.13.3 | 0.52 | 9.3 |
| 2.12.12.2 | 0.14 | 3.6 |
| 2.12.12.4 | 0.14 | 3.0 |
| 2.2.12.15 | 0.13 | N/A |
| 2.2.12.31 | 0.13 | N/A |
| BMK.115 | 0.10 | 3.1 |
| 2.2.5 | 0.14 | 5.8 |
| 2.3.1 | 0.10 | 3.0 |
| 2.3.3 | 0.10 | 2.9 |
| 2.6.19 | 0.16 | 7.1 |
| 2.42.1 | 0.07 | 2.9 |
| 2.42.2 | 0.11 | 2.8 |
| BMK.115 | 0.13 | 3.0 |
| 2.44.2 | 0.49 | 4.9 |
| 2.44.6 | 0.87 | 5.6 |
| 2.52.2 | 1.1 | 1.8 |
| 2.52.4 | 1.0 | 1.8 |
| 2.54.4 | 0.54 | 5.0 |
| 2.54.5 | 0.74 | 6.7 |
| BMK.115 | 0.15 | 2.9 |

2.5 Subcloning

Hybridoma cells of each selected lines were plated in 96-well plates at densities of 0.5, 1 and 5 cell/well. The single clones were picked and tested in binding ELISA. Three subclones of each hybridoma line were selected and frozen.

2.6 Isotype

Antibody Isotype was identified by ELISA. Plates (Nunc) were coated with goat anti-mouse IgG1, anti-mouse IgG2a, anti-mouse IgG2b, anti-mouse IgG3, anti-mouse IgM antibodies at 1 b g/ml overnight at 4° C. After blocking and washing, the hybridoma supernatants were transferred to the coated plates and incubate at room temperature for 1 h. The plates were then incubated with secondary antibody goat anti-mouse kappa HRP or goat anti mouse lambda HRP (Southern Biotech) for 45 min. After washing, TMB substrate was added and the interaction was stopped by 2M HCl. The absorbance at 450 nm was read using a microplate reader (Molecular Device).

2.7 Antibody Purification

Harvested hybridoma supernatants were loaded to Protein A column (MabSelect SuRe, GE) after adjusting pH to 7.0. Antibodies were eluted by Glycine followed with immediately neutralization using 1 M Tris. Antibody concentration was tested by Nano Drop (Thermal-Fisher). The purity of proteins was evaluated by SDS-PAGE (Invitrogen, NuPAGE4%-12% Bis-Tris Gel) and HPLC-SEC (Agilent).

### Example 3: Lead Antibody Characterization

3.1 LDL Uptake Assay

HepG2 or Huh-7 cells were seeded in a 96-well plate at a density of 1×10⁵ cells/well in DMEM medium containing 10% FBS. The plate was kept in a 37° C. incubator overnight. The medium was replaced with DMEM without FBS. Mixture of human PCSK9 and various concentrations of antibodies were added to the cells. The final concentration of PCSK9 was 20 μg/ml. After 1 hour, Bodipy FL-labeled LDL (Invitrogen L-3483) was added to the cells to make a final concentration of 1.5 μg/ml. After incubation in a 37° C. incubator for 3 hours, medium containing LDL in the plate was discarded. The cells were trypsinized and washed twice. LDL-uptake was characterized by the fluorescence of Bodipy FL-labeled LDL in the cells determined by FACS. LDL-uptake restoration rate was calculated following the formula: LDL-uptake restoration (%)=(MFIₛₐₘₚₗₑ−MFI_{LDL+WBP301.AglH})/(MFI_{LDL only}−MF_{ILDL+WBP301.AglH})×100%.

Lead Antibody Characterization

The selected subclones were further evaluated in LDL uptake assay, kinetic affinity test, cross-family binding assay and binning assay. The selection of final candidates was based on binding affinity, blocking activity and cellular LDL-uptake restoration activity.

LDL-uptake assay: the ability of the antibodies to restore the cellular LDL uptake was evaluated using HepG2 cell (see FIG. 3). The IC50 values of LDL-uptake assay are summarized in Table 4. Several antibodies showed 2 digit nanomolar IC50. This result demonstrates that the applied antibodies can efficiently restore the cellular LDL uptake.

TABLE 4
Summary of LDL-uptake assay

| Ab | IC50 (nM) |
|---|---|
| 2.2.5 | 173.7 |
| 2.3.3 | 126.4 |
| 2.6.19 | 209 |
| 2.42.1 | 91 |
| 2.44.2 | 259.4 |
| 2.52.4 | 127.2 |
| 2.54.4 | 151.8 |
| BMK.115 | 54.6 |
| 2.6.6 | 129.8 |
| 2.13.1 | 136.9 |
| 2.12.12.4 | 72.5 |
| 2.3.3 | 79.4 |
| 2.42.1 | 66.8 |
| 2.52.4 | 84.6 |
| BMK.115 | 56.9 |

3.2 Binding Kinetics by SPR

Antibody binding affinity to human and rhesus PCSK9 was detected by SPR assay using Biacore T200 (GE). Each antibody was captured on anti-murine IgG Fc antibody (GE) or anti-human IgG Fc antibody (Genway) immobilized CM5 sensor chip (GE). Human or rhesus PCSK9 at different concentrations were injected over the sensor chip at a flow rate of L/min for an association phase of 180 s, followed by 1200 s dissociation. The chip was regenerated by 2 M MgCl₂ after each binding cycle.

The sensorgrams for blank surface and buffer channel are subtracted from the test sensorgrams. The experimental data was fitted by 1:1 model using Langmiur analysis. Molecular weight of 85 KDa was used to calculate the molar concentration of analyte.

Kinetic affinity by SPR: the affinities of selected clones binding to human PCSK9 were measured by SPR assay. The kₒₙ, k_{off} and K_{D} data are summarized in Table 5. Several antibodies showed nanomolar or sub-nanomolar affinity to human PCSK9.

TABLE 5
Kinetic affinity to human PCSK9

| | Kinetic affinity | | |
|---|---|---|---|
| Antibody | ka (1/Ms) | kd (1/s) | KD (M) |
| BMK.115 | 1.48E+05 | 1.86E−05 | 1.25E−10 |
| 2.2.5 | 2.28E+06 | 3.60E−02 | 1.58E−08 |
| 2.3.3 | 8.28E+05 | 1.45E−03 | 1.75E−09 |
| 2.6.19 | 1.30E+05 | 1.53E−04 | 1.18E−09 |
| 2.42.1 | 8.23E+05 | 2.08E−03 | 2.53E−09 |
| 2.44.2 | 1.80E+05 | 4.70E−03 | 2.61E−08 |
| 2.52.4 | 1.82E+05 | 3.47E−03 | 1.90E−08 |
| 2.54.4 | 1.11E+05 | 1.64E−04 | 1.48E−09 |
| 2.6.6 | 1.37E+05 | 1.48E−04 | 1.08E−09 |
| 2.13.1 | 1.23E+05 | 1.16E−04 | 9.39E−10 |
| 2.12.12.4 | 1.02E+06 | 5.43E−05 | 5.31E−11 |

3.3 Cross-Reactivity to Rhesus PCSK9 by ELISA

ELISA plates (Nunc) were coated with anti-His antibody (Genscript) at 1 μg/ml overnight at 4° C. After blocking and washing, 1 μg/ml rhesus PCSK9-His (Sino Biological) was added and incubated for 1 h. The antibody samples were added to the plates and incubate at room temperature for 1 h. The plates were then washed and subsequently incubated with secondary antibody goat anti mouse-IgG-Fc-HRP (Bethyl) for 45 min. After washing, TMB substrate was added and the interaction was stopped by 2M HCl. The absorbance at 450 nm was read using a microplate reader (Molecular Device).

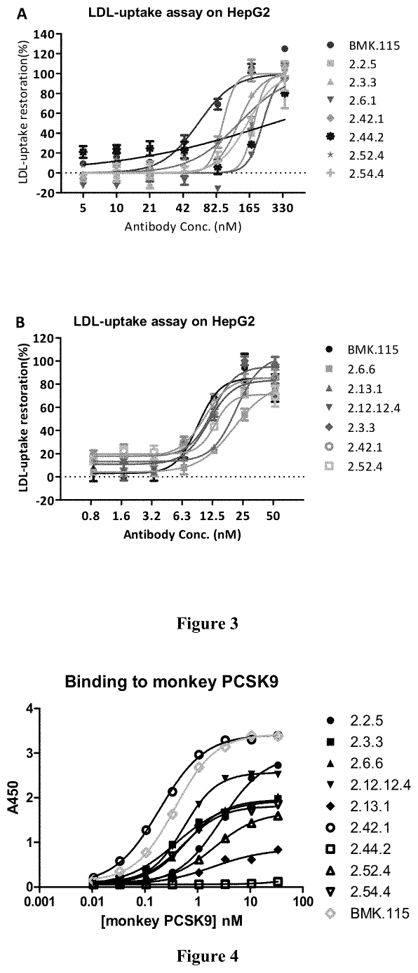

Binding to monkey PCSK9: the affinity of selected antibodies binding to rhesus monkey PCSK9 was measured by ELISA (see FIG. 4). Binding affinity was summarized in Table 6.

TABLE 6
Affinity to monkey PCSK9

| Antibody | KD (nM) |
|---|---|
| 2.2.5 | 2.8 |
| 2.3.3 | 0.3 |
| 2.6.6 | 0.6 |
| 2.12.12.4 | 0.5 |
| 2.13.1 | 1.3 |
| 2.42.1 | 0.2 |
| 2.44.2 | N/A |
| 2.52.4 | 1.7 |
| 2.54.4 | 0.5 |
| BMK.115 | 0.4 |

### Example 4: Generation of Humanized Antibody

4.1 Hybridoma Sequencing

Extract RNA from hyridoma cell using Trizol reagent (Invitrogen-15596018). cDNA was amplified using 5′-RACE kit (Takara-28001488), followed by PCR amplification using 3′-degenerated primers and 3′-adaptor primers (ExTaq: Takara-RR001B). PCR fragments was inserted into pMD 18-T vector (Takara-D101C) and sent for sequencing (Shanghai Biosune). Variable region sequences (amino acid sequences and nucleic acid sequences) of selected antibodies 2.6.6 and 2.12.12.4 and the humanized versions thereof are shown as SEQ ID NOs: 36-51.

4.2 Generation of Recombinant Chimeric Antibody

The V-region DNA of each murine antibody was cloned into a pcDNA3.3 vector containing human constant region gene. HEK293 cell was transfected with plasmids that encode antibody heavy and light chains. Supernatant from transfected cells was harvested by removing cells and filtration. Antibodies were purified by Protein A column (MabSelect SuRe, GE) and buffer exchanged into PBS. Antibody concentration was detected by Nanodrop. Purity was evaluated by SDS-PAGE (Invitrogen, NuPAGE4%-12% Bis-Tris Gel) and HPLC-SEC (Agilent).

4.3 Humanization

“Best Fit” approach was used to humanize antibody light and heavy chains. For light chains amino acid sequences of corresponding V-genes were blasted against in-house human germline V-gene database. The sequence of humanized VL-gene was derived by replacing human CDR sequences in the top hit with mouse CDR sequences using Kabat CDR definition. For heavy chains 4 humanized sequences were derived. First sequence was derived as for light chain. Three additional sequences were created by blasting mouse frameworks against human germline V-gene database. Frameworks were defined using extended CDR definition where Kabat CDR1 was extended by 5 amino acids at N-terminus. Top three hits were used to derive sequences of humanized VH-genes. Humanized genes were back-translated, codon-optimized for mammalian expression, and synthesized by GeneArt Costum Gene Synthesis (Life Technologies). Synthetic genes were re-cloned into IgG expression vector, expressed, and purified.

The humanized antibody was re-evaluated for binding and blocking assay, LDL uptake assay and kinetic affinity test.

4.3.1 Binding and Blocking Activities of Humanized Antibody

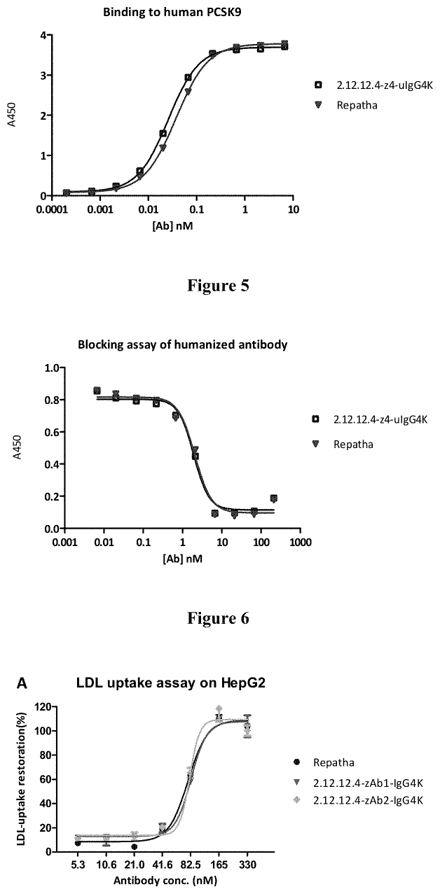

Binding and blocking activity of humanized antibody was detected by ELISA (FIGS. 5 and 6). The humanized antibody 2.12.12.4-z4-uIgG4k showed similar binding and blocking activities compared with its parental murine antibody. The binding EC50 and inhibition IC50 data is summarized in Table 7. The results also demonstrate that the binding and blocking activity of 2.12.12.4-z4-uIgG4k is comparable with Repatha (evolocumab).

TABLE 7
Summary of binding and blocking assay of humanized antibody

| | Binding | Blocking |
|---|---|---|
| Antibody | EC50 (nM) | IC50 (nM) |
| 2.12.12.4-z4-uIgG4K | 0.026 | 1.9 |
| Repatha | 0.038 | 2.1 |

4.3.2 Kinetic Affinity of Humanized Antibody

Affinity of humanized antibodies 2.12.12.4-z4-uIgG4k and 2.6.6-z4-uIgG1k were measured by Biacore. The affinities to human and monkey PCSK9 are summarized in table 8.

TABLE 8
Kinetic affinity of humanized antibodies

| | Kinetic affinity to human PCSK9 | | |
|---|---|---|---|
| Antibody | ka (1/Ms) | kd (1/s) | KD (M) |
| 2.12.12.4-z4-uIgG4k | 9.84E+05 | 2.67E−05 | 2.71E−11 |
| 2.6.6-z4-uIgG1k | 2.22E+04 | 4.69E−05 | 2.11E−09 |
| | Kinetic affinity to monkey PCSK9 | | |
| Antibody | ka (1/Ms) | kd (1/s) | KD (M) |
| 2.12.12.4-z4-uIgG4k | 8.00E+05 | 3.03E−04 | 3.79E−10 |
| 2.6.6-z4-uIgG1k | 1.99E+04 | 3.78E−06 | 1.90E−10 |

4.3.3 LDL-Uptake Assay of Humanized Antibody

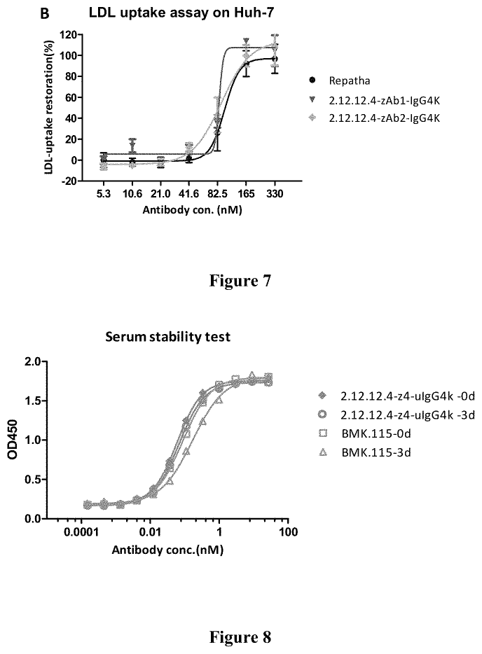

Humanized antibodies were evaluated in LDL-uptake assay in HepG2 and Huh-7 cells (FIG. 7) using wild type PCSK9. Humanized 2.12.12.4 antibody and Repatha showed similar restoration activity of cellular LDL-uptake in HepG2 and Hut-7 cells. The IC50 value of each antibody was summarized in Table 9.

TABLE 9
LDL-uptake assay of humanized antibody

IC50 (nM)

| Antibody | HepG2 | Huh-7 |
|---|---|---|
| 2.12.12.4-z1-IgG4K | 83.5 | 86.2 |
| 2.12.12.4-z2-IgG4K | 80.8 | 90.9 |
| Repatha | 77.6 | 98.2 |

4.3.4 Serum Stability of Humanized Antibody

Antibodies were incubated in freshly isolated human serum (serum content>95%) at 37° C. for 0, 1, 3, 7, 14 days, respectively. After incubation at 37° C., samples were rapidly frozen in dry-ice-ethanol bath and kept at −80° C. The samples were rapidly thawed before stability test. The plates were coated with streptavidin in Na₂CO₃/NaHCO₃ (pH 9.2) buffer at 4° C. overnight. The plates were washed with 0.1% Tween-PBS once before being blocked with 2% BSA/PBS. Biotin-labeled PCSK9 was added and incubated for 1 hr. After washing, then diluted serum samples were transferred to the plates and incubated for 1 hr at room temperature. Goat anti-human-HRP antibody were added to the wells and incubated for 1 hr. After washing, TMB substrate was added and the interaction was stopped by 2 M HCl. The absorbance at 450 nm was read using a microplate reader (Molecular Device).

The antibody binding to human PCSK9 was tested by ELISA after incubation in human serum at 37° C. The binding of antibody 2.12.12.4-z4-uIgG4k after incubation of 3 days did not show significant difference from the pre-incubation sample (see FIG. 8). Therefore, antibody 2.12.12.4-z4-uIgG4k is stable in human serum for 3 days at 37° C.

4.3.5 Antibody Engineering

The humanized antibody 2.12.12.4-z4-uIgG4k was further engineered for developability purpose. Two amino acid residues in variable region were mutated (as shown as 2.12.12.4-v2-z4-uIgG4k). The engineered antibody sequence is displayed in SEQ ID NOs: 44-47. The binding activity of engineered antibody was evaluated by SPR affinity test. The kinetic affinity (Table 10) revealed that the engineered 2.12.12.4-v2-z4-uIgG4k retains the binding activity of its parental clone.

TABLE 10
Kinetic affmity of engineered antibody lead
2.12.12.4-v2-z4-uIgG4k (D30E, N85D) affinity by SPR

| Antigen | ka (1/Ms) | kd (1/s) | KD (M) |
|---|---|---|---|
| human PCSK9 | 1.02E+06 | 4.32E−05 | 4.23E−11 |
| monkey PCSK9 | 5.58E+05 | 2.91E−04 | 5.22E−10 |

4.3.6 Production of Humanized Antibodies Form Transient Transfected Cell Line

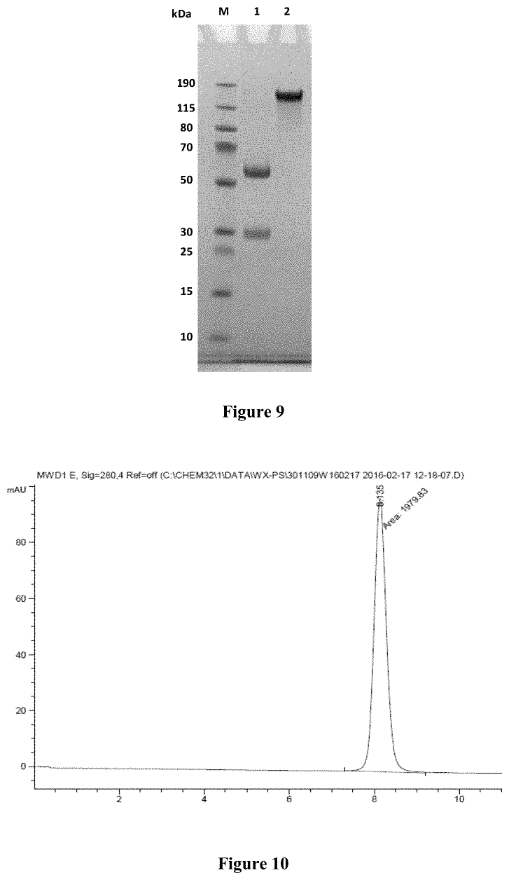

Humanized antibody 2.12.12.4-z4-uIgG4k migrates with the apparent molecular mass of 25 kDa and 55 kDa in SDS-PAGE under reducing condition corresponding to light chain and heavy chain (see FIG. 9). The main band under non-reducing condition is the whole IgG with M.W. of ˜150 KD. The purity is 100% as determined by HPLC-SEC (see FIG. 10). Endotoxin is lower than 0.5 EU/mg.

### Example 5: Animal Study

A total of 4 female cynomolgus monkeys, approximately 3 to 4 years old and weighing 2.5 to 3.5 kg, at dosing initiation. 4 female monkeys were randomly assigned to 4 groups of 1 female/group. Four groups of 1 female monkey each received 3 or 10 mg/kg of Repatha, or 2.12.12.4-z4-uIgG4K (not engineered) by a single dose intravenous injection. The first dosing day was defined as Day 1. The animals of each group were observed for 36 days following dosing

| | Dose^{a} | | | Numbering |
|---|---|---|---|---|
| Group/Label | | Dose | Volume | of Animals |
| color | Test Article | (mg/kg) | (mL/kg) | Female |
| 1/White | Repatha | 3 mg/kg | 0.06 | 1501 |
| 2/Green | Repatha | 10 mg/kg | 0.2 | 2501 |
| 3/Yellow | 2.12.12.4-z4-uIgG4K | 3 mg/kg | 0.56 | 11501 |
| 4/Red | 2.12.12.4-z4-uIgG4K | 10 mg/kg | 1.86 | 12501 |

Note:
In this report, “dose level” and “dosage” are used interchangeably.
^{a}Doses represent active ingredient unless specified otherwise by the Sponsor.

5.1 Single-Dose Efficacy in Non-Human Primates

LDL-C and HDL-C concentration in monkey serum was tested on Roche/Hitachi cobas c systems using LDLC and HDLC3 kits (Roche). Total cholesterol (TCHO) was tested by cholesterol FS kit (DiaSys).

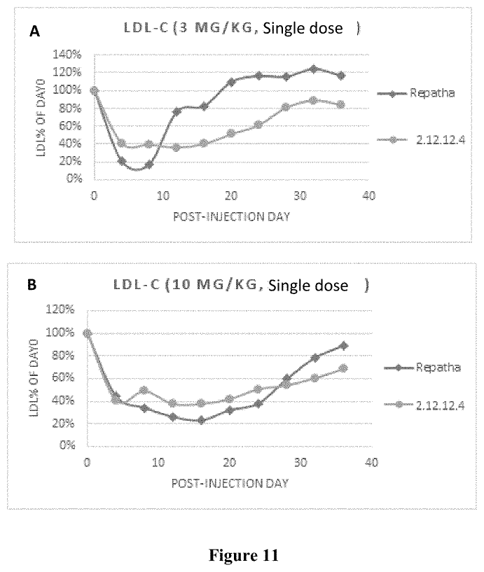

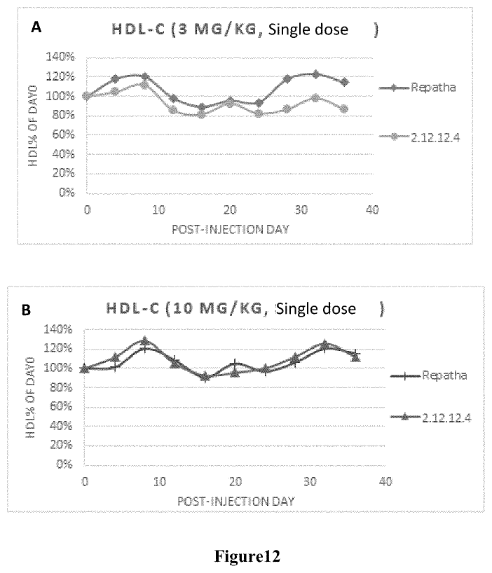

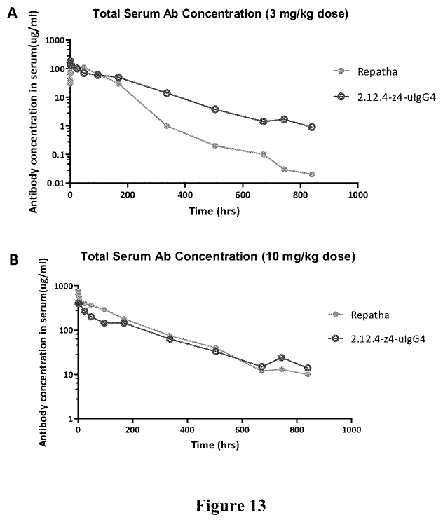

LDL-C lowering effect of antibody 2.12.12.4-z4-uIgG4k in cynomolgus monkey. Administration of Repetha and 2.12.12.4-z4-uIgG4k resulted in a rapid and sustained reduction in LDL-C and total cholesterol (TCHO) at 3 mg/kg and 10 mg/kg in cynomolgus monkeys (see FIG. 11). High Density Lipoprotein cholesterol (HDL-C) was generally well maintained in the monkeys treated with Repetha or 2.12.12.4-z4-uIgG4k at 3 mg/kg and 10 mg/kg (see FIG. 12).

Percentage reduction of LDL was up to 80% and 750% in Repatha 3 mg/kg and 10 mg/kg dose groups, respectively, compared with predose value. 2.12.12.4-z4-uIgG4k yielded significant reductions in LDL-C up to 65% in 3 mg/kg and 10 mg/kg dose groups, respectively. The maximum reduction was reached on day 8-16. Reductions in LDL-C were sustained longer period for 2.12.12.4-z4-uIgG4k treated animals in both 3 mg/kg and 10 mg/kg dose groups compared with Repatha treated groups.

5.2 Pharmacokinetics (PK) Study

To determine systemic exposure (TK), serum concentrations of Repatha and 2.12.12.4-z4-uIgG4K were determined. Blood samples were collected from all available monkeys at 0 (predose), 0.5, 1, 2, 4, 24, 48, 96, 168, 336, 504, 672, 744, and 840 hours postdose.

Approximately 2 mL of blood was collected from the animals via a cephalic or a femoral vein. Blood was collected into appropriately labeled tubes without anticoagulant. The tubes were placed at room temperature for at least 30 min, and serum was obtained within 2 hour of collection by centrifugation at 2000×g and ˜4° C. for 10 minutes. Serum was transferred into uniquely labeled polypropylene tubes and frozen in the upright position immediately over dry ice and stored in a freezer set to maintain ≤−60° C.

The serum samples were rapidly thawed before PK test. The plates were coated with polyclonal goat anti-human antibody in Na₂CO₃/NaHCO₃ buffer at 4° C. overnight. The plates were washed with 0.1% Tween-PBS once before being blocked with 2% BSA/PBS. Diluted cynomolgus serum samples were transferred to the plates and incubated for 1 hr at room temperature. Biotin-labeled goat anti-human IgG antibody and streptavidin-HRP were added to the wells and incubated for 1 hr respectively. The OD value at 450 nm of each well was read after the addition of the substrate and stop solution. The concentrations of antibodies in serum samples were determined by the standard curves.

TK parameter values, including (where data allows), but not necessarily limited to, the initial serum concentrations (C₀), and the area under the serum concentration vs time curve (AUC) from time zero to 840 hours postdose AUC₀₋₈₄₀ₕ, were determined using a validated WinNonlin program (Pharsight, Version 6.2.1). AUC₀₋₈₄₀ₕ was calculated using the linear up/log down trapezoidal rule by noncompartmental methods from drug treated animals only. Serum concentrations below the lower limit of quantification (BLQ) were set to zero for TK parameters calculations.

The antibody concentration in monkey serum was tested by ELISA (FIG. 13). The C₀ and AUC₀₋₈₄₀ₕ for Repatha and 2.12.12.4-z4-uIgG4k following once single IV injection at 3 or 10 mg/kg to female monkeys are presented below in Table 11. The half-life of each antibody was also listed in Table 11. Antibody 2.12.12.4-z4-uIgG4k exhibited longer half-life than Repatha in both doses.

As the dosage increased from 3 mg/kg to 10 mg/kg, the systemic exposure (AUC₀₋₈₄₀ₕ and/or C₀) to Repatha and 2.12.12.4-z4-uIgG4k increased dose-proportionally.

TABLE 11
Summary of PK data

| | Dose | C₀ | AUC_{0-840 h} | T_{1/2} |
|---|---|---|---|---|
| Analyte | (mg/kg) | (ug/mL) | (h*ug/mL) | (h) |
| Repatha | 3 | 30 | 14000 | 62 |
| | 10 | 802 | 88900 | 145.2 |
| 2.12.12.4-z4-uIgG4k | 3 | 160 | 18700 | 114.3 |
| | 10 | 400 | 63800 | 196.3 |

5.3 Immunogenicity

Blood was collected from the animals at 0 (predose), 336, 672 and 840 hours postdose via a cephalic or a femoral vein.

The plates were coated with Repatha or 2.12.12.4-z4-uIgG4K in Na₂CO₃/NaHCO₃ buffer at 4° C. overnight. The plates were washed with 0.1% Tween-PBS once before being blocked with 2% BSA/PBS. PBS-diluted cynomolgus serum samples were transferred to the plates and incubated for 1 hr at room temperature. After washing, goat anti-cynomolgus IgG-HRP antibody (no cross-interaction with human IgG) was added. The OD value at 450 nm of each well was read after the addition of the substrate and stop solution.

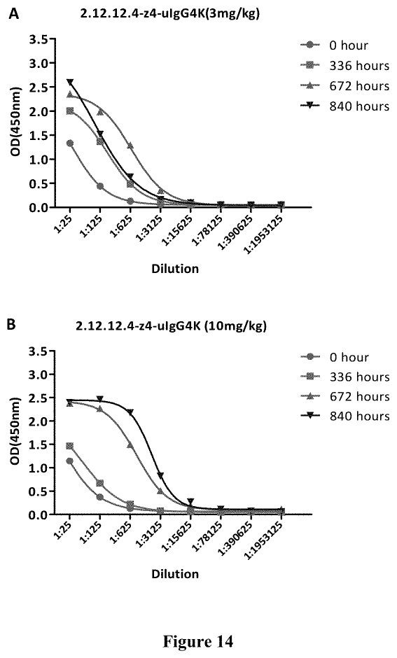

The immunogenicity test results of 2.12.12.4-z4-uIgG4k are shown in FIG. 14. The titers of anti-drug antibody (ADA) against 2.12.12.4-z4-uIgG4k in monkey serum increased at 336, 672, 840 hours post dose.

5.4 Toxicity

Mortality/Moribundity: The health status of each animal was reported twice a day during the study, once in the morning and once in the afternoon, except on animal release and the day of in-life completion where animals were examined once.

There were no unscheduled deaths during the course of the study.

Detailed observations: Detailed observations were conducted once during pretest for all animals (including spare animals), once on dosing day (2±0.5 hours post dose), and once weekly thereafter during the study for all study animals.

There were no test article-related clinical signs observed during the in-life phase.

Cage side observations: Cage side observation was conducted daily during pretest from Day −2 for all animals (including spare animals), once predose on Day 1, and twice daily during the dosing day (within 30 minutes, and at approximately 6±0.5 hours post dose) and once daily during the recovery phase. Cage side observation was not conducted if a detailed observation was scheduled at the same time slot.

Body weights: Each animal was weighed once during pretest for all animals, once on Day 1 prior to dosing, and once weekly thereafter for study animals.

No test article-related findings were observed on body weight, and all changes were considered within biological variation.

Food consumption: Food consumption was estimated daily for all animals 2 days prior to dose initiation and throughout the dosing day and observation period. Daily food evaluation was assessed by visual inspection for overall appetite (the documentation was consist of whether the animal was eating or not).

There were no test article-related changes in food consumption.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. An isolated antibody or an antigen binding fragment thereof, comprising a heavy chain CDR sequence selected from the group consisting of: SEQ ID NOs: 1, 3, 5, 13, 15, and 17, and/or a light chain CDR sequence selected from the group consisting of: SEQ ID NOs: 7, 9, 11, 19, 21, 23 and 25.

2. (canceled)

3. The antibody or an antigen binding fragment thereof of claim 1, comprising a heavy chain variable region selected from the group consisting of:
a) a heavy chain variable region comprising SEQ ID NO: 1, SEQ ID NO: 3, and/or SEQ ID NO: 5; and
b) a heavy chain variable region comprising SEQ ID NO: 13, SEQ ID NO: 15, and/or SEQ ID NO: 17 and/or a light chain variable region selected from the group consisting of:
c) a light chain variable region comprising SEQ ID NO: 7, SEQ ID NO: 9, and/or SEQ ID NO: 11;
d) a light chain variable region comprising SEQ ID NO: 19, SEQ ID NO: 21, and/or SEQ ID NO: 23; and
e) a light chain variable region comprising SEQ ID NO: 25, SEQ ID NO: 21, and/or SEQ ID NO: 23.

4. (canceled)

5. The antibody or an antigen binding fragment thereof of claim 1, comprising:
a) a heavy chain variable region comprising SEQ ID NO: 1, SEQ ID NO: 3, and/or SEQ ID NO: 5; and a light chain variable region comprising SEQ ID NO: 7, SEQ ID NO: 9, and/or SEQ ID NO: 11;
b) a heavy chain variable region comprising SEQ ID NO: 13, SEQ ID NO: 15, and/or SEQ ID NO: 17; and a light chain variable region comprising SEQ ID NO: 19, SEQ ID NO: 21, and/or SEQ ID NO: 23; or
c) a heavy chain variable region comprising SEQ ID NO: 13, SEQ ID NO: 15, and/or SEQ ID NO: 17; and a light chain variable region comprising SEQ ID NO: 25, SEQ ID NO: 21, and/or SEQ ID NO: 23.

6. The antibody or an antigen binding fragment thereof of claim 1, comprising a heavy chain variable region selected from the group consisting of: SEQ ID NO: 36, SEQ ID NO: 40, SEQ ID NO: 44, SEQ ID NO: 48 and the homologue sequences of at least 80% sequence identity thereof, and/or a light chain variable region selected from the group consisting of: SEQ ID NO: 38, SEQ ID NO: 42, SEQ ID NO: 46, SEQ ID NO: 50 and the homologue sequences of at least 80% sequence identity thereof.

7. (canceled)

8. The antibody or an antigen binding fragment thereof of claim 1, comprising:
a) a heavy chain variable region comprising SEQ ID NO: 36 and a light chain variable region comprising SEQ ID NO: 38;
b) a heavy chain variable region comprising SEQ ID NO: 40 and a light chain variable region comprising SEQ ID NO: 42;
c) a heavy chain variable region comprising SEQ ID NO: 44 and a light chain variable region comprising SEQ ID NO: 46;
d) a heavy chain variable region comprising SEQ ID NO: 48 and a light chain variable region comprising SEQ ID NO: 50; or
e) a heavy chain variable region and a light chain variable region of at least 80% sequence identity to a), b), c) or d).

9-15. (canceled)

16. The antibody or antigen binding fragment thereof of any claim 1, which is a humanized monoclonal antibody.

17-19. (canceled)

20. An antibody or an antigen binding fragment thereof, which competes for the same epitope with the antibody or the antigen binding fragment thereof of claim 1.

21. The antibody or antigen-binding fragment thereof of claim 1, which is a camelized single domain antibody, a diabody, a scFv, an scFv dimer, a BsFv, a dsFv, a (dsFv)2, a dsFv-dsFv′, an Fv fragment, a Fab, a Fab′, a F(ab′)2, a ds diabody, a nanobody, a domain antibody, or a bivalent domain antibody.

22. The antibody or antigen-binding fragment thereof of claim 1, further comprising an immunoglobulin constant region.

23. The antibody or antigen-binding fragment thereof of claim 1, further comprising a conjugate.

24. An isolated polynucleotide encoding the antibody or an antigen binding fragment thereof of claim 1.

25. A vector comprising the isolated polynucleotide of claim 24.

26. A host cell comprising the vector of claim 25.

27. A method of expressing the antibody or antigen-binding fragment thereof of claim 1, comprising culturing the host cell of comprising a vector comprising an isolated polynucleotide encoding the antibody or an antigen binding fragment thereof under the condition at which the polynucleotide is expressed.

28. A kit comprising the antibody or antigen-binding fragment thereof of claim 1.

29. A method of treating a disease or condition mediated by PCSK9 in an individual, comprising: administering a therapeutically effective amount of antibody or antigen-binding fragment thereof of claim 1 to the individual.

30. The method of claim 29, wherein the individual has been identified as having a disorder or a condition likely to respond to a PCSK9 inhibitor.

31. The method of claim 30, wherein the individual has been identified as upregulated level of serum LDL cholesterol, total cholesterol and/or non-HDL cholesterol in a test biological sample from the individual.

32. A pharmaceutical composition comprising the antibody or antigen-binding fragment thereof of claim 1 and one or more pharmaceutically acceptable carriers.

33. A method of treating a condition in a subject that would benefit from upregulation of immune response, comprising administering a therapeutically effective amount of the antibody or antigen-binding fragment thereof of claim 1, to the subject.

34. The method of claim 33, wherein the subject has upregulated level of serum LDL cholesterol, total cholesterol and/or non-HDL cholesterol.

35. (canceled)

36. The method of claim 33, wherein the condition is cardiovascular diseases, inflammatory diseases, or infectious diseases.

37. The method of claim 36, wherein the infectious disease is sepsis.


SEQUENCE LISTING (51 sequences)

SEQ ID NO: 1 - protein, 5 aa, Mus musculus
SYWIH

SEQ ID NO: 2 - DNA, 15 bp, Mus musculus
agctactggatacac

SEQ ID NO: 3 - protein, 17 aa, Mus musculus
ELNPSDGRTNYNEKFKN

SEQ ID NO: 4 - DNA, 51 bp, Mus musculus
gagcttaatcctagcgacggtcgtactaactacaatgagaagttcaagaac

SEQ ID NO: 5 - protein, 11 aa, Mus musculus
SNFYGSSSFAN

SEQ ID NO: 6 - DNA, 33 bp, Mus musculus
tcgaatttctacggtagtagctcctttgctaac

SEQ ID NO: 7 - protein, 10 aa, Mus musculus
SASSSVSYMN

SEQ ID NO: 8 - DNA, 30 bp, Mus musculus
agtgccagctcaagtgtaagttacatgaac

SEQ ID NO: 9 - protein, 7 aa, Mus musculus
GISNLAS

SEQ ID NO: 10 - DNA, 21 bp, Mus musculus
ggtatatccaacctggcttct

SEQ ID NO: 11 - protein, 9 aa, Mus musculus
QQRSSYPPT

SEQ ID NO: 12 - DNA, 27 bp, Mus musculus
cagcaaaggagtagttacccacccacg

SEQ ID NO: 13 - protein, 5 aa, Mus musculus
NYVMH

SEQ ID NO: 14 - DNA, 15 bp, Mus musculus
aactatgttatgcac

SEQ ID NO: 15 - protein, 17 aa, Mus musculus
YINPYNDYIKYNEKFKG

SEQ ID NO: 16 - DNA, 51 bp, Mus musculus
tatattaatccttataatgattatattaagtacaatgagaagttcaaaggc

SEQ ID NO: 17 - protein, 14 aa, Mus musculus
QRYYGYRPYYAMDY

SEQ ID NO: 18 - DNA, 42 bp, Mus musculus
cagaggtactacggttatagaccctactatgctatggactat

SEQ ID NO: 19 - protein, 15 aa, Mus musculus
RASESVDGYGNVFMH

SEQ ID NO: 20 - DNA, 45 bp, Mus musculus
agagccagtgaaagtgttgatggttatggcaatgtttttatgcac

SEQ ID NO: 21 - protein, 7 aa, Mus musculus
LASNLES

SEQ ID NO: 22 - DNA, 21 bp, Mus musculus
cttgcatctaacctggaatct

SEQ ID NO: 23 - protein, 9 aa, Mus musculus
QQNNKDPWT

SEQ ID NO: 24 - DNA, 27 bp, Mus musculus
cagcaaaataataaggatccgtggacg

SEQ ID NO: 25 - protein, 15 aa, Artificial Sequence
  Synthetic
RASESVEGYGNVFMH

SEQ ID NO: 26 - DNA, 45 bp, Artificial Sequence
  Synthetic
cgggcctcagaaagtgtggagggatacgggaacgtgttcatgcac

SEQ ID NO: 27 - DNA, 15 bp, Artificial Sequence
  Synthetic
aactacgtgatgcac

SEQ ID NO: 28 - DNA, 51 bp, Artificial Sequence
  Synthetic
tatattaacccctacaacgactacatcaagtacaatgagaagtttaaaggg

SEQ ID NO: 29 - DNA, 42 bp, Artificial Sequence
  Synthetic
cagcggtactacggctacaggccctactacgccatggattat

SEQ ID NO: 30 - DNA, 15 bp, Artificial Sequence
  Synthetic
agctactggatccac

SEQ ID NO: 31 - DNA, 51 bp, Artificial Sequence
  Synthetic
gagctgaaccccagtgatgggaggacaaattacaacgagaagtttaagaac

SEQ ID NO: 32 - DNA, 33 bp, Artificial Sequence
  Synthetic
tcaaacttctatggcagcagtagctttgccaac

SEQ ID NO: 33 - DNA, 30 bp, Artificial Sequence
  Synthetic
tccgccagttcatcagtgagttacatgaat

SEQ ID NO: 34 - DNA, 21 bp, Artificial Sequence
  Synthetic
ggaatctccaaccttgccagc

SEQ ID NO: 35 - DNA, 27 bp, Artificial Sequence
  Synthetic
cagcagaggagcagctatccacccacc

SEQ ID NO: 36 - protein, 120 aa, Mus musculus
QVQLQQPGAEVVKPGASVTVSCKASGYTFTSYWIHWVMQRPGQGLEWIGELNPSDGRTNYNEKFKNKATLTVDTSSSTVYMQLSSLTSEDSAVYYCARSNFYGSSSFANWGQGTLVTVSA

SEQ ID NO: 37 - DNA, 360 bp, Mus musculus
caggtccaactgcagcagcctggggctgaagtggtgaagcctggggcttcagtgacggtgtcctgcaaggcttctggctacaccttcaccagctactggatacactgggtgatgcagagacctggacaaggccttgagtggattggagagcttaatcctagcgacggtcgtactaactacaatgagaagttcaagaacaaggccacactgactgtagacacatcgtccagtacagtctacatgcaactcagcagcctgacatctgaggactctgcggtctattactgtgcaagatcgaatttctacggtagtagctcctttgctaactggggccaagggactctggtcactgtctctgca

SEQ ID NO: 38 - protein, 106 aa, Mus musculus
EILLTQSPAIIAASPGEKVTITCSASSSVSYMNWYQQKPGSSPILWIYGISNLASGVPARFSGSGSGTSFSFTINNMEAEDVATYYCQQRSSYPPTFGGGTKLEIK

SEQ ID NO: 39 - DNA, 318 bp, Mus musculus
gaaattttgctcacccagtctccagcaatcatagctgcatctcctggggagaaggtcaccatcacctgcagtgccagctcaagtgtaagttacatgaactggtatcagcagaaaccaggatcctcccccatactatggatctatggtatatccaacctggcttctggagttcctgctcgcttcagcggcagtgggtctgggacatctttctctttcacaatcaacaacatggaggctgaagatgttgccacttattactgtcagcaaaggagtagttacccacccacgttcggaggggggaccaagctggaaataaaa

SEQ ID NO: 40 - protein, 123 aa, Mus musculus
EVQLQQSGPELVKPGASVKMSCEASGYTFTNYVMHWVKQKPGQGLEWIGYINPYNDYIKYNEKFKGKATLTSDKSSSTTYLEVSSLTSEDSAVYYCGRQRYYGYRPYYAMDYWGQGTSVTVSS

SEQ ID NO: 41 - DNA, 369 bp, Mus musculus
gaggtccagctgcagcagtctggacctgagctggtaaagcctggggcctcagtgaagatgtcctgcgaggcttctggatacacattcactaactatgttatgcactgggtgaagcagaagcctgggcagggccttgagtggattggatatattaatccttataatgattatattaagtacaatgagaagttcaaaggcaaggccacactgacttcagacaaatcctccagtacaacctacctggaagtcagcagcctgacctctgaggactctgcggtctattactgtggaagacagaggtactacggttatagaccctactatgctatggactattggggtcagggaacctcagtcaccgtctcctca

SEQ ID NO: 42 - protein, 111 aa, Mus musculus
NIVLTQSPASLAVSLGQRATISCRASESVDGYGNVFMHWYQQKPGQPPKLLIYLASNLESGVPDRFSGSGSRTDFTLTIDPVEADDAATYYCQQNNKDPWTFGGGTKLEIK

SEQ ID NO: 43 - DNA, 333 bp, Mus musculus
aacattgtgctgacccaatctccagcttctttggctgtgtctctaggtcagagggccaccatttcctgcagagccagtgaaagtgttgatggttatggcaatgtttttatgcactggtaccagcagaaaccaggacagccacccaaactcctcatctatcttgcatctaacctggaatctggggtccctgacaggttcagtggcagtgggtctaggacagacttcaccctcaccattgatcctgtggaggctgatgatgctgcaacttattactgtcagcaaaataataaggatccgtggacgttcggtggaggcaccaagctggaaatcaaa

SEQ ID NO: 44 - protein, 123 aa, Artificial Sequence
  Synthetic
QVQLVQSGAEVKKPGSSVKVSCEASGYTFTNYVMHWVRQAPGQGLEWMGYINPYNDYIKYNEKFKGKATITADKSTSTAYMELSSLRSEDTAVYYCGRQRYYGYRPYYAMDYWGQGTLVTVSS

SEQ ID NO: 45 - DNA, 369 bp, Artificial Sequence
  Synthetic
caagtgcagctcgtgcagtctggagcagaagtgaagaagcctggatcatccgtcaaggtgagttgcgaggcttccgggtacacattcaccaactacgtgatgcactgggtcagacaggctccagggcaggggcttgagtggatggggtatattaacccctacaacgactacatcaagtacaatgagaagtttaaagggaaagccaccatcactgccgacaagagcaccagcacagcctacatggagctgtccagcctgaggagcgaggatacagccgtctactactgtggccgccagcggtactacggctacaggccctactacgccatggattattggggccagggcactttggtgacagtcagctcc

SEQ ID NO: 46 - protein, 111 aa, Artificial Sequence
  Synthetic
DIVLTQSPASLAVSPGQRATITCRASESVEGYGNVFMHWYQQKPGQPPKLLIYLASNLESGVPARFSGSGSGTDFTLTINPVEADDTANYYCQQNNKDPWTFGGGTKVEIK

SEQ ID NO: 47 - DNA, 333 bp, Artificial Sequence
  Synthetic
gacatcgtcttgacccagtctcctgcttcccttgcagtgtcccctgggcaaagagctactattacctgccgggcctcagaaagtgtggagggatacgggaacgtgttcatgcactggtaccagcagaagcccggacagccaccaaagctgctcatctacctggccagcaacctcgagagcggcgtgcccgccaggtttagcgggagcggctccgggactgatttcacactgacaattaatcccgtcgaggccgacgatacagccaactattactgtcagcagaacaacaaggacccctggacatttggcggcgggaccaaagtcgagatcaag

SEQ ID NO: 48 - protein, 120 aa, Artificial Sequence
  Synthetic
QVQLVQSGAEVKKPGASVKVSCKASGYTFTSYWIHWVRQAPGQGLEWMGELNPSDGRTNYNEKFKNKATMTVDTSTSTVYMELSSLRSEDTAVYYCARSNFYGSSSFANWGQGTLVTVSS

SEQ ID NO: 49 - DNA, 360 bp, Artificial Sequence
  Synthetic
caggtgcaacttgtgcagtccggagcagaagtgaagaagccaggggcttcagtgaaggtctcctgcaaggcttccggatacaccttcaccagctactggatccactgggtcagacaggcccctgggcagggcttggagtggatgggcgagctgaaccccagtgatgggaggacaaattacaacgagaagtttaagaacaaagccaccatgaccgtcgacacatccacaagcacagtgtacatggagctctccagcctgcgcagcgaggacactgccgtctactactgtgcccggtcaaacttctatggcagcagtagctttgccaactggggccaggggactctggtgactgtgtcttct

SEQ ID NO: 50 - protein, 106 aa, Artificial Sequence
  Synthetic
DIQMTQSPSSLSASVGDRVTITCSASSSVSYMNWYQQKPGKAPKLLIYGISNLASGVPSRFSGSGSGTDFTFTISSLQPEDIATYYCQQRSSYPPTFGGGTKVEIK

SEQ ID NO: 51 - DNA, 318 bp, Artificial Sequence
  Synthetic
gacatccaaatgacccagagcccttcttccctctccgcttctgtcggagatcgggtcacaatcacatgctccgccagttcatcagtgagttacatgaattggtaccagcagaagcctgggaaggcacccaagctgctgatctacggaatctccaaccttgccagcggcgtgccaagcagattctccggcagcgggagcgggacagatttcacctttacaattagctccttgcagcccgaagacattgccacttactactgtcagcagaggagcagctatccacccacctttggcggcgggactaaagtggagattaag